(19) United States
(12) Patent Application Publication
(10) Pub. No.: US 2009/0169520 A1
(43) Pub. Date: Jul. 2, 2009

(54) Compounds, Pharmaceutical Compositions and Therapeutic Methods of Preventing and Treating Diseases and Disorders Associated With Amyloid Fibril Formation

(75) Inventors: Hermona Soreq, Jerusalem (IL); Sophie Diamant, Mevasseret Zion (IL)

Correspondence Address: MARTIN D. MOYNIHAN d/b/a PRTSI, INC., P.O. BOX 16446, ARLINGTON, VA, 22215, US

(73) Assignee: YISSUM RESEARCH DEVELOPMENT COMPANY OF THE HEBREW UNIVERSITY OF JERUSALEM, Jerusalem (IL)

(21) Appl. No.: 10/585,695

(22) PCT Filed: Jan. 9, 2005
(86) PCT No.: PCT/IL2005/000028

§ 371 Date: Aug. 20, 2008

(51) Int. Cl. A61K 35/12; C07K 14/00; A61K 38/16; A61K 31/7088

(52) U.S. Cl. 424/93.7; 530/324; 514/12; 436/501; 514/44

(57) ABSTRACT

Compounds, pharmaceutical compositions and methods for prevention and/or reversal of amyloid fibril formation and treating amyloid-related disorders are provided. Also provided a method of limiting and/or reducing inflammation.

### FIELD AND BACKGROUND OF THE INVENTION

The present invention relates to compounds, pharmaceutical compositions and therapeutic methods of preventing and/or inhibiting fibril formation and more particularly to methods of preventing and/or treating amyloid—related diseases and disorders. The present invention further relates to methods of treating inflammations.

Proper protein folding is a crucial step required for normal functioning and turnover of proteins. However, various factors such as stress, specific genetic mutations and certain infections may induce a cascade of yet incompletely understood processes leading to conformational changes or misfolding of proteins and consequently to their abnormal accumulation as amyloid fibrils. Such conformational changes often involve the conversion from an α-helix configuration to a β-pleated sheet structure. These structural rearrangements, followed by nucleation, polymerization, aggregation and fibril formation, play a central role in the pathogenesis of most neurodegenerative diseases, such as Alzheimer's, Huntington's, Parkinson's and prion diseases, as well as at least eight of the polyglutamine—related disorders [Kaytor, M. D. and Warren, S. T. (1999) J. Biol. Chem. 274(53): 37507-10] and various amyloidosis syndromes (e.g., Multiple myeloma, Chronic inflammatory disease, Rheumatoid arthritis, Tuberculosis, Skin and lung abscesses, Cancer, Hodgkin's disease, Hemodialysis for CRF, Heredofamilial amyloidosis, Familial Mediterranean Fever and Familial amyloid polyneuropathy).

For example, Alzheimer's disease (AD) is characterized by the formation and progressive deposition of insoluble amyloid fibrils within the cerebral cortex. The key constituent of these amyloid deposits has been identified as a 39-43-amino acid long polypeptide, the β-amyloid peptide (AD). Once deposited as dense amyloid plaque cores, the peptide becomes highly resistant to further proteolysis and causes dystrophy of the surrounding nerve cells [Knauer et al. (1992) Proc. Natl. Acad. Sci. U.S.A. 89(16): 7437-41; Nordstedt et al. (1994) J. Biol. Chem. 269(49): 30773-6]. However, it is still unclear whether the amyloid fibrils themselves or the soluble oligomers of Aβ are the main neurotoxic species that contribute to neurodegeneration and dementia present in Alzheimer's disease or other amyloidosis—related disorders (De Felice F G, et al., 2004, FASEB J. 18: 1366-72).

Several studies aiming at identifying therapeutic approaches for preventing amyloid fibril formation have suggested the use of beta-sheet breaker such as N,N′-bis(3-hydroxyphenyl)pyridazine-3,6-diamine (RS-0406) to reverse amyloid beta-induced cytotoxicity (Nakagami Y, et al., 2002, Br. J. Pharmacol. 137: 676-82), the use of N-methylated derivatives to inhibit toxicity and protofibril formation in the amyloid-beta peptide beta(25-35) (Doig A J, et al., 2002, Biochem. Soc. Trans. 30(4): 537-42), the mechanical unzipping of amyloid beta-fibrils (Kellermayer M S, et al., 2004, J. Biol. Chem. Epub ahead of print), the use of curcumin as an anti-inflammatory agent which suppresses amyloid accumulation (Yang F et al., 2004, J. Biol. Chem. December 7; Epub ahead of print), the use of a monoclonal antibody specific to the C-terminal 92-99 of beta(2)ₘ (Motomiya Y, et al., 2005, Kidney Int. 67: 314-20) and the use of nonsteroidal anti-inflammatory drugs (NSAIDs) to stabilize Transthyretin (TTR) tetramers (Miller S R, et al., 2004, Lab Invest. 84: 545-52).

Butyrylcholinesterase (BChE, EC 3.1.1.8) is the primary circulating cholinesterase, abundant in serum and present at synapses and neuromuscular junctions, where it binds the same structural unit as the synaptic variant of acetylcholinesterase (AChE), AChE-S, with which it shares C-terminal sequence homology. Like ACHE, BChE is capable of hydrolyzing acetylcholine (ACh) at the end of each round of pre-synaptic secretion. However, while ACHE has a narrow substrate specificity, BChE exhibits a wide specificity for both substrates and inhibitors.

Prior studies have shown that acetylcholinesterase (ACHE) co-localizes with the Aβ peptides present in the brain of Alzheimer's patients [Inestrosa, N. C. et al. (1996a) Mol. Psychiatry. 1(5): 359-61; Inestrosa, N. C. et al. (1996b) Neuron 16(4): 881-91;] and that amyloid P complexes including ACHE are far more neurotoxic than Aβ aggregates alone [Alvarez et al. (1998) J. Neurosci. 18(9):3213-23]. Moreover, AChE, but not BChE, was found to promote aggregation of amyloid complexes [Inestrosa, 1996b (Supra)].

Despite advances in the field, there is still a great need to identify a suitable therapeutic agent for preventing amyloid fibril formation.

### SUMMARY OF THE INVENTION

While reducing the present invention to practice, the present inventors have uncovered that BChE and more so BChE derived peptides are capable of preventing and/or reversing amyloid fibril formation and thus can be used to prevent and/or treat amyloidosis—related disorders and diseases. It was further found that BChE can prevent or reduce inflammation.

According to one aspect of the present invention there is provided a method of identifying a BChE derived peptide capable of preventing and/or reversing amyloid fibril formation comprising contacting the BChE derived peptide with an amyloid precursor protein and a β-sheet—responsive dye and measuring a fluorescence intensity resulting from the β-sheet—responsive dye prior to and following the contacting the BChE derived peptide with the amyloid precursor protein, wherein delayed or reduced increase in the fluorescence intensity following the contacting the BChE derived peptide with the amyloid precursor protein is indicative of an ability of the peptide to prevent amyloid fibril formation. This is a high throughput method which is readily automateable and which can be used to test, for example, within a short time period each one of the peptides represented by SEQ ID NOs:8-20302, all are BChE derived peptides.

According to further features in preferred embodiments of the invention described below, the β-sheet—responsive dye is a benzothiazole dye.

According to still further features in the described preferred embodiments the β-sheet—responsive dye is Thioflavin T.

According to still further features in the described preferred embodiments the Thioflavin T is provided at a concentration range of 0.5-1.5 μM.

According to still further features in the described preferred embodiments the Thioflavin T is provided at a concentration of about 1 μM. As used herein throughout the term “about” refers to ±10%.

According to still further features in the described preferred embodiments the amyloid precursor protein is selected from the group consisting of Transthyretin, Amyloid beta protein, Amyloid beta (1-40), Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

According to still further features in the described preferred embodiments the Amyloid beta (1-40) is provided at a concentration in the range of 20-50 μM.

According to still further features in the described preferred embodiments the Amyloid beta (1-40) is provided at a concentration of about 33 μM.

The method described above can be used to identify and point out BChE derived peptides capable of preventing and/or reversing amyloid fibril formation.

Hence, according to another aspect of the present invention there is provided a BChE derived peptide capable of preventing and/or reversing amyloid fibril formation. In presently preferred embodiments, the BChE derived peptide is selected from the group consisting of SEQ ID NOs:1 and 8-20302.

According to yet another aspect of the present invention there is provided a pharmaceutical composition comprising as an active ingredient BChE or a BChE derived peptide, the peptide being capable of preventing and/or reversing amyloid fibril formation and a pharmaceutically acceptable carrier.

According to yet another aspect of the present invention there is provided a method of treating an individual having or being predisposed to a disease or disorder associated with amyloid fibril formation, the method comprising administering to the individual a therapeutically effective amount of BChE or BChE derived peptide, thereby treating the individual having or being predisposed to a disease or disorder associated with amyloid fibril formation.

According to further features in the described preferred embodiments of the invention described below, the BChE derived peptide is selected from the group consisting of SEQ ID NO:1 and 8-20302.

According to still further features in the described preferred embodiments the disease or disorder associated with amyloid fibril formation is selected from the group consisting of a neurodegenerative disease, a disorder associated with systemic amyloidosis, a disorder associated with localized amyloidosis, a prion disease and/or a polyglutamine disorder.

According to still further features in the described preferred embodiments the neurodegenerative disease is selected from the group consisting of Alzheimer's disease, Huntington's disease and Parkinson's disease.

According to still further features in the described preferred embodiments the disorder associated with systemic amyloidosis is selected from the group consisting of Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy.

According to still further features in the described preferred embodiments the disorder associated with localized amyloidosis is selected from the group consisting of Senile cardiac amyloidosis, Senile cerebral amyloidosis, Endocrine tumors, Medullary carcinoma of thyroid, Type II diabetes and Pancreatic islets β-cells.

According to still further features in the described preferred embodiments the prion disease is selected from the group consisting of Creutzfeldt-Jakob disease (CJD), spongioform enchephalopathies (TSE's), mad cow disease, Gerstmann-Straussler-Scheinker disease (GSS) and Kuru.

According to still further features in the described preferred embodiments the polyglutamine disorder is selected from the group consisting of Huntington's disease (HD), Spinal and Bulbar Muscular Atrophy (SBMA), DentatoRubral and PallidoLuysian Atrophy (DRPLA), spinocerebellar ataxia type 1 (SCA1), spinocerebellar ataxia type 2 (SCA2), Spinocerebellar ataxia type-3 (SCA3; Machado-Joseph Disease), Spinocerebellar ataxia type 7 (SCA7) and Spinocerebellar ataxia type 17 (SCA17).

According to still further features in the described preferred embodiments the amyloid is a protein selected from the group consisting of Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

According to still further features in the described preferred embodiments the active ingredient is formulated in a therapeutically effective amount providable at a dose range of 0.1-1000 micromol per kg body weight.

According to still further features in the described preferred embodiments the active ingredient is formulated in a therapeutically effective amount providable at a dose range of 1-100 micromol per kg body weight.

According to still further features in the described preferred embodiments the active ingredient is formulated in a therapeutically effective amount providable at a dose range of 5-50 micromol per kg body weight.

According to still another aspect of the present invention there is provided a method of preventing and/or reversing amyloid fibril formation in a tissue of an individual comprising increasing a level of BChE or a BChE derived peptide being capable of preventing and/or reversing amyloid fibril formation in the tissue, thereby preventing and/or reversing amyloid fibril formation therein.

According to yet another aspect of the present invention there is provided a method of treating an individual having or being predisposed to a disease or disorder associated with amyloid fibril formation, the method comprising increasing a level of BChE or a BChE derived peptide in a tissue susceptible to the amyloid fibril formation of the individual, thereby treating the individual having or being predisposed to a disorder associated with amyloid fibril formation.

According to further features in preferred embodiments of the invention described below, increasing a level of BChE or a BChE derived peptide being capable of preventing and/or reversing amyloid fibril formation in the tissue is effected by at least one approach selected from the group consisting of (a) expressing in cells of the individual an exogenous polynucleotide encoding the BChE or the BChE derived peptide; (b) increasing expression of endogenous BChE in the individual; (c) increasing endogenous BChE activity in the individual; (d) administering BChE or the BCHE derived peptide to the individual; and (e) administering to the individual cells expressing the BChE or the BChE derived peptide.

According to still further features in the described preferred embodiments of the invention further described below, the BChE is as set forth in SEQ ID NO:2.

According to still further features in the described preferred embodiments the exogenous polynucleotide encoding the BChE or the BChE derived peptide is derived from SEQ ID NO:7.

According to an additional aspect of the present invention there is provided a method of limiting or reducing an inflammatory reaction in an individual, comprising increasing an expression level and/or activity of BChE in the individual, thereby limiting or reducing the inflammatory reaction in the individual.

According to still further features in the described preferred embodiments of the invention further described below, the inflammatory reaction is mediated by circulating acetylcholine.

According to still further features in the described preferred embodiments, the individual is subjected to a surgery, stress or a trauma.

According to still further features in the described preferred embodiments the inflammatory reaction is mediated by at least one pro-inflammatory cytokine selected from the group consisting of IL-1, IL-1α, IL-1β, IL-1ss, IL-6, IL-8, IL-10, IL-12, IL-18 and TNFα.

According to still further features in the described preferred embodiments increasing an expression level and/or activity of BChE in the individual is effected by at least one approach selected from the group consisting of (a) expressing in cells of the individual an exogenous polynucleotide encoding at least a functional portion of BChE; (b) increasing expression of endogenous BChE in the individual; (c) increasing endogenous BChE activity in the individual; (d) administering an exogenous polypeptide including at least a functional portion of BChE to the individual; and (e) administering cells expressing BChE into the individual.

The present invention successfully addresses the shortcomings of the presently known configurations by providing novel compounds, compositions a method of preventing and/or reversing amyloid fibril formation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-e are graphs depicting the effects of BChE and ACHE on amyloid fibril formation. Aβ [1-40], at a final concentration of 33 μM, was incubated at 30° C. with shaking, in the presence of 1 μM Thioflavin T (ThT). The kinetics of change in ThT fluorescence was followed with time. Where indicated, the incubation solution contained BChE (FIG. 1a) and/or ACHE-S (FIG. 1b) or both enzymes at the noted micromolar concentrations and at the constant ratio of 1:100 ACHE or BChE to Aβ (FIGS. 1c-d). Shown are representative findings from a series of 12-16 experiments in each case. Note that while BChE suppresses amyloid formation, ACHE accelerates such fibrillation. Also note the longer time scale prior to the onset of detectable fibrillation for BChE as compared with ACHE. FIG. 1e-a graph depicting the effect of AChE on amyloid formation. Note that increasing the doses of AChE results in increased fibril yields and decreased lag period until fibril formation initiates.∥

FIGS. 2a-b depict the overall effect of BChE on amyloid formation. FIG. 2a is a histogram showing the rate of amyloid formation in the presence or absence of ACHE and/or BChE. Note the significant effect of BChE in reducing the rate of amyloid formation even in the presence of ACHE. FIG. 2b is a schematic illustration summarizing the inhibitory effect of BChE on amyloid fibril formation which counteracts the effect generated by ACHE.

FIGS. 3a-d depict the effect of the synthetic BSP and ASP peptides on amyloid formation. FIG. 3a illustrates the sequence and homology of the BSP41, ASP23, ASP40 and ASP63 peptides. Homologous residues between BSP41 and ASP40 are marked by asterisks, partial homologies are marked with dots. |FIG. 3b-a graph depicting the effect of BSP41 peptide on amyloid fibril formation. Aβ at 33 μM was incubated at 30° C. with 1 μM ThT and the noted micromolar concentrations of BSP41. Shown are the rates of fibril formation for each set of conditions. FIG. 3c—a histogram depicting the rate of amyloid formation (average rates ±SE) for the cumulative data of each protein and peptide tested, derived from the time curves of changes in ThT fluorescence. Note that BSP41, but not ASP23, ASP40, or ASP63 significantly attenuates fibril formation. FIG. 3d—a 3-D model depicting the globular structure of BChE. Note the large distance between the Peripheral Anionic binding Site (PAS) and the C-terminal domain (CT), both of which are highlighted in green.

FIG. 4 depicts the circular dichroism (CD) spectra of the BSP and ASP peptides. Shown are the circular dichroism (CD) spectra of 1·10⁻⁴ M BSP41 dissolved in HIFP and 1·10⁻⁴ M ASP40, ASP23 and ASP63 in aqueous solutions. Note the characteristic features of α-helix in BSP41, ASP40, or ASP63 as compared with the random coil seen in ASP23.

FIGS. 5a-e depict the comparative three-dimensional molecular modeling of BSP and ASP. FIGS. 5a-b depict the molecular modeling of BSP (FIG. 5a; purple) and ASP (FIG. 5b; sky blue). Note that both peptides are amphipathic helices, i.e.; each helix can be divided into a polar and non-polar side (as demonstrated by the yellow broken line). BSP's division into sharp two distinguished sides is imperfect, since it is disturbed by a tryptophan residue (shown in sticks and colored by element), while ASP's amphipathicity is intact, as shown by the arginine residue (also shown in sticks and colored by element) in the parallel position to the BSP tryptophan. FIGS. 5c-d depict cross-sections of the BSP (FIG. 5c) and ASP (FIG. 5d) helices. Residues were presented as circles (hydrophilic), diamonds (hydrophobic), triangles (potentially negatively charged), or pentagons (potentially positively charged). Hydrophobicity is color coded from green (the most hydrophobic residue) to yellow (zero hydrophobicity), with the green:yellow ratio decreasing proportionally. Hydrophilic residues are coded red (pure red being the most hydrophilic [uncharged] residue) to white, with red:white decreasing proportionally. Potentially charged residues are colored light blue. Note that division by polarity but not by charge or pH of these helices is the only feasible one, as is highlighted by the purple division line. FIG. 5e—is an alignment between the BSP and ASP sequences.|

FIG. 6 is a graph illustrating the effect of BChE in preventing amylin amyloid fibril formation. Amylin at a concentration of 20 μM was incubated with Thiofalvin T (at a concentration of 1 μM) and the formation of amyloid fibril was measured by following the Thiofalvin T fluorescence intensity [excitation (450 nm); emission (485 nm)] in the presence or absence of 0.24 μM BChE (purified from pooled human plasma). Note that the addition of BChE increases the lag time for amylin fibrillation from 30 minutes to 110 minutes.

### DESCRIPTION OF THE PREFERRED EMBODIMENTS

The present invention is of compounds, pharmaceutical compositions and therapeutic methods of preventing amyloid fibril formation and treating or preventing diseases or disorders associated therewith. More specifically the present invention is of BChE derived peptides, pharmaceutical compositions containing BChE and/or BChE derived peptides and therapeutic methods of using BChE and/or BChE derived peptides in prevention and/or treatment of diseases and disorders associated with amyloid fibril formation, such as, but not limited to, neurodegenerative diseases, prion diseases and polyglutamine disorders.

The principles and operation of the method of preventing and/or reversing amyloid fibril formation according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Proper protein folding is crucial for normal protein function and turnover. However, in many cases (e.g., neurodegenerative diseases, prion diseases and polyglutamine disorders) specific proteins are subjected to conformational changes or misfolding which often involve the conversion from an α-helix configuration to a β-pleated sheet structure. Such conformational changes lead to the abnormal accumulation of the misfolded proteins in the form of amyloid fibrils and plaques. Formation of amyloid fibrils has been observed in neurodegenerative diseases (e.g., Alzheimer's disease, Parkinson's disease), polyglutamine diseases [Huntington's disease, Spino-Cerebellar Ataxia (SCA)] and prion diseases [e.g., Kuru, Creutzfeldt-Jakob disease (CJD) spongioform enchephalopathies (TSE's), mad cow disease, and Gerstmann-Straussler syndrome (GSS)].

Prior attempts to prevent amyloid fibril formation suggested the use of beta-sheet breaker such as N,N′-bis(3-hydroxyphenyl)pyridazine-3,6-diamine (RS-0406) to reverse amyloid beta-induced cytotoxicity (Nakagami Y, et al., 2002, Br. J. Pharmacol. 137: 676-82), the use of N-methylated derivatives to inhibit toxicity and protofibril formation in the amyloid-beta peptide beta (25-35) (Doig A J, et al., 2002, Biochem. Soc. Trans. 30(4): 537-42), the mechanical unzipping of amyloid beta-fibrils (Kellermayer M S, et al., 2004, J. Biol. Chem. Epub ahead of print), the use of curcumin as an anti-inflammatory agent which suppresses amyloid accumulation (Yang F et al., 2004, J. Biol. Chem. December 7; Epub ahead of print), the use of a monoclonal antibody specific to the C-terminal 92-99 of beta(2)ₘ (Motomiya Y, et al., 2005, Kidney Int. 67: 314-20) and the use of nonsteroidal anti-inflammatory drugs (NSAIDs) to stabilize Transthyretin (TTR) tetramers (Miller S R, et al., 2004, Lab Invest. 84: 545-52).

While reducing the present invention to practice, the present inventors have uncovered that BChE and more so BChE derived peptides are capable of preventing and/or reversing amyloid fibril formation and thus can be used to prevent and/or treat amyloidosis—related disorders and diseases. It was further found that BChE can prevent or reduce inflammation.

As used herein the phrase “preventing and/or reversing amyloid fibril formation” refers to inhibiting the formation of, avoiding the formation of, delaying the formation of and/or limiting the extent of the formation of amyloid fibrils, as well as, disforming, reducing the level of and/or eliminating preformed amyloid fibrils.

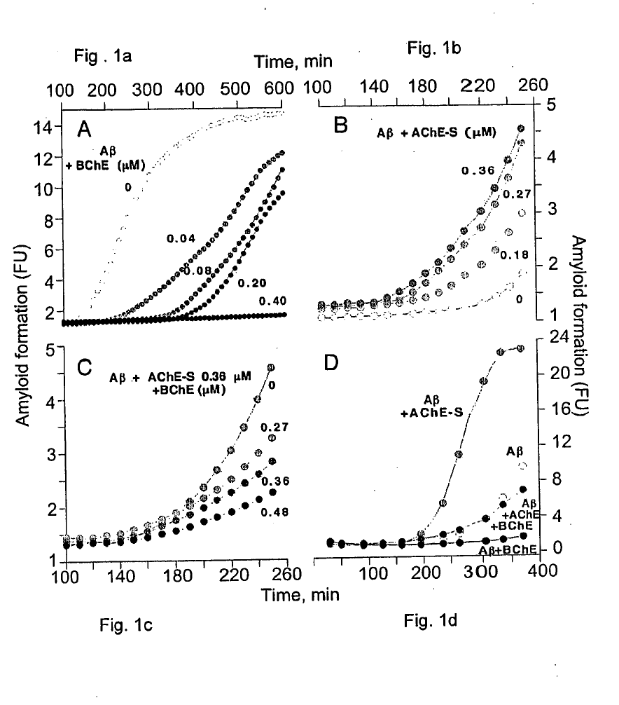

As is shown in FIGS. 1-3 and Table 7 and as is described in Example 1 of the Examples section which follows, the BChE enzyme (SEQ ID NO:2), at a concentration of 30 μg/ml, as well as the synthetic BSP41 peptide (SEQ ID NO:1, amino acids 562-602 of Human BChE which is the C-terminal region of Human BChE), which mimics the C-terminus of human BChE, at a concentration of 2 μg/ml, were capable of completely attenuating Aβ fibrillation following 400 minutes of incubation as determined by change of Thioflavin T (ThT) fluorescence. These results indicate that BChE and BChE derived peptides can be used to prevent the formation of and/or dis-stabilize amyloid fibrils.

As used herein the term “BChE” refers to Butyrylcholinesterase, which is also known as Acylcholine acylhydrolase, Choline esterase II, Butyrylcholine esterase and/or Pseudocholinesterase. Butyrylcholinesterase (BCHE, EC 3.1.1.8) is the primary circulating cholinesterase, abundant in serum and present at synapses and neuromuscular junctions. BChE is capable of hydrolyzing acetylcholine (ACh) at the end of each round of pre-synaptic secretion and exhibits a wide specificity for both substrates and inhibitors.

Hence, according to one aspect of the present invention there is provided a method (assay) of identifying a BChE derived peptide capable of preventing and/or reversing amyloid fibril formation. The method according to this aspect of the invention comprises contacting the BChE derived peptide with an amyloid precursor protein and a β-sheet—responsive dye and measuring a fluorescence intensity resulting from the β-sheet—responsive dye prior to and following contacting the BChE derived peptide with the amyloid precursor protein, wherein delayed or reduced increase in the fluorescence intensity following contact formation between the BCHE derived peptide with the amyloid precursor protein is indicative of an ability of the peptide to prevent amyloid fibril formation.

Contacting according to this aspect of the present invention is effected by means of mixing, shaking or dissolving the BChE derived peptide with the amyloid precursor protein and the β-sheet responsive dye. Preferably, contacting is effected by shaking at a shaking speed of 50-300 rpm, more preferably at a shaking speed of 200 rpm.

According to preferred embodiments of the present invention, contacting is effected for a time period of at least 5 minutes, more preferably, at least 10 minutes, more preferably, at least 15 minutes, more preferably, at least 20 minutes, more preferably, at least 25 minutes, more preferably, at least 30 minutes, more preferably, at least 40 minutes, more preferably, at least 50 minutes, more preferably, at least 60 minutes, more preferably, at least 2 hours, more preferably, at least 4 hours, more preferably, at least 6 hours, most preferably, at least 8 hours.

Measuring the fluorescence intensity according to this aspect of the present invention is preferably effected using a Spectro-fluorometer (e.g., Tecan, Maennedorf, Switzerland). It will be appreciated that measuring can be effected at any given time prior to, during or following contacting the BChE derived peptide. In order to obtain a basal level of the fluorescent intensity generated by the β-sheet—responsive dye, measuring is effected both prior to the addition of the BChE derived peptide and at time intervals following the addition of the BChE derived peptide. The time intervals for measuring the fluorescent intensity may vary depending on the dye used. According to preferred embodiments of the present invention, such time intervals are at least every 60 minutes, more preferably, at least every 50 minutes, more preferably, at least every 40 minutes, more preferably, at least every 30 minutes, more preferably, at least every 20 minutes, more preferably, at least every 10 minutes, most preferably, at least every 5 minutes.

It will be appreciated by one of ordinary skills in the art that this is a high throughput screening method which is readily automateable and which can be used to test, for example, within a short time period each one of the peptides represented by SEQ ID NOs:8-20302, all are BChE derived peptides, for its ability to prevent and/or reverse amyloid fibril formation. In a presently preferred embodiment of the present invention the β-sheet—responsive dye is a benzothiazole dye, such as, but not limited to Thioflavin T. The Thioflavin T is preferably provided at a concentration range of 0.5-1.5 μM, most preferably the Thioflavin T is provided at a concentration of about 1 μM.

As used herein, the phrase “amyloid fibril” refers to the intra- or extracellular tissue deposits, in one or more tissue or organs, of fibril protein material which is generically termed amyloid. The amyloid is distinguished grossly by a starch-like staining reaction with iodine, microscopically by its extracellular distribution and tinctorial and optical properties when bound to Congo red or Thioflavin T, or by its capacity to bind and induce fluorescence in bound Thioflavin T and by its protein fibril structure as shown by electron microscopy and X-ray crystallography. Amyloid fibrils are formed by conformation changes which lead to misfolding of the amyloid precursor protein, such as a conformation conversion from an α-helix configuration to a β-pleated sheet structure. Thus, amyloid fibrils initiate from an innoculum of misfolded proteins which further facilitates fibril formation around it (Reviewed in Lachmann H J and Hawkins P N, 2003. Nephron Clin. Pract. 94: c85-8). The protein precursor of the amyloid fibril of the present invention is, for example, Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

The amyloid precursor protein used in the assay method described herein can be any amyloid precursor proteins listed above. Preferably, the amyloid precursor protein used in the assay method is Amyloid beta (1-40) and it is provided in the assay method at a concentration in the range of 20-50 μM, preferably about 33 μM.

As stated above, it will be appreciated by the skilled artisan that the method described above can be used to identify and point out BChE derived peptides capable of preventing and/or reversing amyloid fibril formation using automated high throughput installations.

Hence, according to another aspect of the present invention there is provided a BChE derived peptide capable of preventing and/or reversing amyloid fibril formation. In presently preferred embodiments, the BChE derived peptide is selected from the group consisting of SEQ ID NOs:1 and 8-20302.

As used herein throughout the phrase “BChE derived peptide” means any peptide sequence of 5, 6, 7, 8, 9, 10, 11, 12, 13, 14, 15 or more, e.g., 16-20, 21-30, 31-40 amino acids, either natural, digest or synthetic that naturally forms a part of a polypeptide at least 60%, at least 70%, at least 80%, at least 90%, at least 95%, at least 96%, at least 97%, at least 98%, at least 99% homologous (similar+identical) to a BChE polypeptide as set forth in SEQ ID NO:2 or 20303-20314 as determined using the BlastP software of the National Center of Biotechnology Information (NCBI) using default parameters. The phrase “BChE derived peptide” further reads on functional homologs of all of the above peptides, which functional homologs can include naturally occurring or non-natural amino acids exhibiting the functional activity of preventing and/or reversing amyloid fibril formation.

Thus, amino acid substitutions can be made in any of the BChE derived peptides described herein. Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative peptide. Generally these changes are done on a few amino acids to minimize the alteration of the functionality of the peptide molecule. However, larger changes may be tolerated in certain circumstances. When small alterations in the characteristics of the peptides of the present invention are desired, substitutions are generally made in accordance with the following Table 1:

TABLE 1

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Table 1, hereinabove. For example, substitutions may be made which more significantly affect: the structure of the peptide backbone in the area of the alteration, for example the alpha-helical or beta-sheet structure; the charge or hydrophobicity of the molecule at the target site; or the bulk of the side chain. The substitutions which in general are expected to produce the greatest changes in the peptide properties are those in which (a) a hydrophilic residue, e.g., seryl or threonyl is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g. phenylalanine, is substituted for (or by) one not having a side chain, e.g. glycine.

Non-natural amino acids can also be used as substituents to naturally occurring amino acids:

Table 2 and 3 below list naturally occurring amino acids (Table 2) and non-conventional or modified amino acids (Table 3) which can be used with the present invention.

TABLE 2

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| isoleucine | Iie | I |
| Leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 3

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl-glycine | Nnbhm | carbamylmethyl(1)glycine | |
| 1-carboxy-1-(2,2-diphenyl | Nmbc | | |
| ethylamino)cyclopropane | | | |

According to preferred embodiments of the present invention, a BChE derived peptide is a peptide that includes at least 5, preferably at least 6 amino acids, preferably, at least 7, more preferably, at least 8, more preferably, at least 9, more preferably, at least 10, more preferably, at least 11, more preferably, at least 12, more preferably, at least 13, more preferably, at least 14, more preferably, at least 15, more preferably, at least 16, more preferably, at least 17, more preferably, at least 18, more preferably, at least 19, more preferably, at least 20, more preferably, at least 21, more preferably, at least 22, more preferably, at least 23, more preferably, at least 24, more preferably, at least 25, more preferably, at least 26, more preferably, at least 27, more preferably, at least 28, more preferably, at least 29, more preferably, at least 30, more preferably, at least 31, more preferably, at least 32, more preferably, at least 33, more preferably, at least 34, more preferably, at least 35, more preferably, at least 36, more preferably, at least 37, more preferably, at least 38, more preferably, at least 39, most preferably, at least 40 amino acids of the BChE polypeptide as set forth by SEQ ID NO:2.

Following is a summary of non-limiting BChE derived peptides which can be used in context of the present invention.

TABLE 4
Human BChE derived peptides

| Length (amino acids) | SEQ ID NOs: |
|---|---|
| 6 | 8-599 |
| 7 | 600-1195 |
| 8 | 1196-1790 |
| 9 | 1791-2384 |
| 10 | 2385-2977 |
| 11 | 2978-3569 |
| 12 | 3570-4171 |
| 13 | 4161-4750 |
| 14 | 4751-5339 |
| 15 | 5340-5927 |
| 16 | 5928-6514 |
| 17 | 6515-7100 |
| 18 | 7101-7685 |
| 19 | 7686-8269 |
| 20 | 8270-8852 |
| 21 | 8853-9434 |
| 22 | 9435-10015 |
| 23 | 10016-10595 |
| 24 | 10596-11174 |
| 25 | 11175-11752 |
| 26 | 11753-12329 |
| 27 | 12330-12905 |
| 28 | 12906-13480 |
| 29 | 13481-14054 |
| 30 | 14055-14627 |
| 31 | 14628-15199 |
| 32 | 15200-15770 |
| 33 | 15771-16340 |
| 34 | 16341-16909 |
| 35 | 16910-17477 |
| 36 | 17478-18044 |
| 37 | 18045-18610 |
| 38 | 18611-19175 |
| 39 | 19176-19739 |
| 40 | 19740-20302 |

Table 4: SEQ ID numbers of human BChE derived peptides are presented according to their size (number of amino acids). The hBChE derived peptides were designed according to the hBChE sequence (SEQ ID NO: 2), SwissProt. Accession No. P06276.

It is hereby reiterated, any of the BChE derived peptides described herein can be tested for it ability in preventing and/or reversing fibril formation using the assay method described hereinabove.

According to yet another aspect of the present invention there is provided a pharmaceutical composition comprising as an active ingredient BChE or a BChE derived peptide, the peptide being capable of preventing and/or reversing amyloid fibril formation; and a pharmaceutically acceptable carrier. Any one or more of the BChE derived peptides described herein and their functional analogs can be used as the active ingredient of the pharmaceutical composition of the present invention.

According to yet another aspect of the present invention there is provided a method of treating an individual having or being predisposed to a disease or disorder associated with amyloid fibril formation. The method according to this aspect of the invention comprises administering to the individual a therapeutically effective amount of BChE or BChE derived peptide, thereby treating the individual having or being predisposed to a disease or disorder associated with amyloid fibril formation.

As used herein, the term “treating” refers to inhibiting or arresting the development of a disease, disorder or condition and/or causing the reduction, remission, or regression of a disease, disorder or condition. Those of skills in the art will understand that various methodologies and assays can be used to assess the development of a disease, disorder or condition and similarly, various methodologies and assays may be used to assess the reduction, remission or regression of a disease, disorder or condition.

As used herein the term “individual” includes both young and old human beings of both sexes it also refers to animals such as live stock animals. The term also encompasses individuals who are at risk to develop the amyloid fibril associated disease or disorder as described hereinabove.

Any one or more of the BChE derived peptides described herein and their functional homologs can be used in the methods of the present invention. While implementing the method of the present invention and according to presently preferred embodiments of the present invention, the BChE or BChE derived peptide is administered to treated individuals in the form of a pharmaceutical composition.

The presently preferred peptides to be used in the therapeutic method and pharmaceutical composition described herein are selected from the group consisting of SEQ ID NO:1 and 8-20302.

Many diseases and disorders can be treated using the therapeutic peptides, pharmaceutical compositions and therapeutic methods of the invention, these diseases and disorders are associated with amyloid fibril formation, such as, for example, neurodegenerative disease, e.g., Alzheimer's disease, Huntington's disease and Parkinson's disease; disorders associated with systemic amyloidosis, such as, but not limited to, Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy (Cardoso I, et al., 2003, FASEB J. 17: 803-9); disorders associated with localized amyloidosis, such as, but not limited to, Senile cardiac amyloidosis, Senile cerebral amyloidosis, Endocrine tumors, Medullary carcinoma of thyroid, Type II diabetes and Pancreatic islets β-cells; prion diseases, such as, but not limited to, Creutzfeldt-Jakob disease (CJD), spongioform enchephalopathies (TSE's), mad cow disease, Gerstmann-Straussler-Scheinker disease (GSS) and Kuru (Guiroy D C, et al., 1994; Acta Neuropathol. (Berl). 87: 526-30). Spino-Cerebellar Ataxia (SCA); and/or polyglutamine disorders, such as, but not limited to, Huntington's disease (HD; Fox J H et al., 2004, J. Neurochem. 91: 413-22), Spinal and Bulbar Muscular Atrophy (SBMA), DentatoRubral and PallidoLuysian Atrophy (DRPLA), spinocerebellar ataxia type 1 (SCA1; Emamian E S et al., Neuron. 2003 May 8; 38(3):375-87), spinocerebellar ataxia type 2 (SCA2; Satterfield T F, et al., 2002, Genetics, 162: 1687-702), Spinocerebellar ataxia type-3 (SCA3; Machado-Joseph Disease; Berke S J et al., 2004, J. Neurochem. 89: 908-18; Chow M K et al., 2004, J. Biol. Chem. 279: 47643-51), Spinocerebellar ataxia type 7 (SCA7; Helmlinger D et al., 2004, J. Neurosci. 24: 1881-7), and Spinocerebellar ataxia type 17 (SCA17; Tsuji S, 2004, Arch Neurol. 61: 183-4; Oda M, et al., 2004, Arch Neurol. 61: 209-12; Ross C A, 2002, Neuron. 35: 819-22). Any one of these diseases and disorders is associated with amyloid fibril formation of one or more of the following proteins: Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

The classification of amyloidosis is based upon the tissue distribution of amyloid deposits (local or systemic amyloidosis), the absence or presence of preexisting disease (primary or secondary amyloidosis) and the chemical type of amyloid protein fibril. By convention, amyloid fibril types are designated by two letters: A for amyloid followed by a letter for the chemical type. There are two, chemically distinct, major types of amyloid protein fibrils designated AL (amyloid light chain) and AA (non-immunoglobulin amyloid associated), as well as several minor types. AL fibrils associated mainly with multiple myeloma are related to monoclonal immunoglobulin light chains synthesized by abnormal plasma cells. AA fibrils associated mainly with chronic inflammatory diseases are related to the non-immunoglobulin amyloid associated (AA) protein and its serum precursor (SAA), an acute phase reactant synthesized by liver cells.

Tables 5 and 6, hereinbelow present the classifications of various amyloidosis—related disorders.

TABLE 5
Classification of amyloidosis: Systemic Amyloidosis

| Clinical | | Amyloid | |
|---|---|---|---|
| Classification | Associated Condition | Fibril Type | Precursor |
| Primary or | Multiple myeloma | AL | Ig lambda |
| Secondary | | | (or kappa |
| | | | chains) |
| Secondary | Chronic inflammatory | AA | SAA |
| | disease, Rheumatoid | | |
| | arthritis, Tuberculosis, | | |
| | Skin and lung abscesses | | |
| Secondary | Cancer, Hodgkin's | AA | SAA |
| | disease | | |
| Secondary | Hemodialysis for CRF | Beta2-m | Beta-2-m |
| Primary | Heredofamilial | AA | SAA |
| | amyloidosis, Familial | Transthyretin | Transthyretin |
| | Mediterranean Fever, | | |
| | Familial amyloid | | |
| | polyneuropathy | | |

Table 5: Classification of systemic amyloids is presented. CRF = chronic renal failure; Beta-2-m = beta 2-microglobulin (a normal serum protein and a component of MHC class I molecules); Transthyretin = a normal serum protein that transports thyroxin and retinol (vitamin A) and is deposited in a variant form.

TABLE 6
Classification of amyloidosis: Localized amyloidosis

| Associated | | |
|---|---|---|
| Condition | Amyloid Fibril Type | Precursor |
| Senile cardiac | Transthyretin | Transthyretin |
| amyloidosis | | |
| Senile cerebral | Amyloid beta protein | Amyloid precursor |
| amyloidosis: | | protein (APP) |
| Alzheimer's | | |
| disease | | |
| Endocrine tumors, | Procalcitonin | Calcitonin |
| Medullary | | |
| carcinoma of | | |
| thyroid | | |
| Type II diabetes | IAPP (Amylin) | IAPP (Amylin) |
| Pancreatic islets | | |
| β-cells | | |

Table 6: Classification of localized amyloids is presented.

The therapeutically effective amount preferably used in context of the therapeutic methods of the present invention is 0.1-1000 micromol of the BChE derived peptide per kg body weight, more preferably, 1-100 micromol per kg body weight, yet more preferably, 5-50 micromol per kg body weight.

According to still another aspect of the present invention there is provided a method of preventing and/or reversing amyloid fibril formation in a tissue of an individual. The method according to this aspect of the present invention comprises increasing a level of BChE or a BChE derived peptide being capable of preventing and/or reversing amyloid fibril formation in the tissue, thereby preventing and/or reversing amyloid fibril formation therein.

The term “tissue” as used herein refers to part of an organism consisting of an aggregate of cells structured and arranged to perform at least one biological or physiological function, such as brain tissue, retina, skin tissue, hepatic tissue, pancreatic tissue, bone tissue, cartilage tissue, joint tissue, lymph node tissue, connective tissue, blood tissue, muscle tissue, cardiac tissue, brain tissue, vascular tissue, renal tissue, pulmonary tissue, gonadal tissue, hematopoietic tissue and fat tissue. Amyloid fibrils and amyloid fibrils associated diseases and disorders may be associated with any one of the above listed tissues.

According to yet another aspect of the present invention there is provided a method of treating an individual having or being predisposed to a disease or disorder associated with amyloid fibril formation. The method according to this aspect of the present invention comprises increasing a level of BChE or a BChE derived peptide in a tissue susceptible to amyloid fibril formation in that individual, thereby treating the individual having or being predisposed to a disorder associated with amyloid fibril formation.

As is further detailed below, increasing a level of BChE or a BChE derived peptide being capable of preventing and/or reversing amyloid fibril formation is effected by at least one approach selected from the group consisting of (a) expressing in cells of the individual an exogenous polynucleotide encoding the BChE or the BChE derived peptide; (b) increasing expression of endogenous BChE in the individual; (c) increasing endogenous BChE activity in the individual; (d) administering BChE or the BChE derived peptide to the individual; and (e) administering to the individual cells expressing the BChE or the BChE derived peptide.

Increasing the level of BChE or a BChE derived peptide capable of preventing and/or reversing amyloid fibril formation can be effected in many ways, such as by upregulating expression of endogenous BChE or by introducing into the tissue exogenous BChE, portions thereof or polynucleotide sequences encoding either.

Upregulation of endogenous BChE can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with inhibitors and/or substrates and the like). For example, upregulating the endogenous expression of BChE can be achieved by administering at least one natural or synthetic substrate and/or ligand of a transcription factor controlling BChE gene expression in amounts sufficient to induce a natural response of overproduction of BChE. However, since peripheral site ligands activate the hydrolytic activity of BChE (Glick, 2003), it is possible to modulate this activity by co-administration of the ligand with specific BChE inhibitors.

Thus, an agent capable of upregulating BChE may be any compound which is capable of increasing the transcription and/or translation of an endogenous DNA or mRNA encoding the BChE and thus increasing endogenous BChE activity.

Upregulating expression of BChE via exogenous polypeptide or polynucleotide sequences can be effected by introducing into cells of the tissue an exogenous polynucleotide sequence designed and constructed to express at least a portion of the BChE. Accordingly, the exogenous polynucleotide sequence may be a DNA or RNA sequence encoding a BChE molecule, capable of preventing and/or reversing amyloid fibril formation.

BChE sequences have been cloned from various sources including human (Prody, PNAS, 1987; Arpagaus, M. et al., 1990, Biochemistry 29: 124-131; GenBank Accession No. P06276; SEQ ID NO:2), rat (Nakahara T, et al., 2003, Urol. Res. 31: 223-226; GenBank Accession No. NP_{—}075231; SEQ ID NO:20303), mouse (Rachinsky T L, et al., 1990, Neuron 5: 317-327; GenBank Accession No. NP_{—}033868, Q03311; SEQ ID NO:20304), cat (Bartels C F, et al., 2000, Biochem. Pharmacol. 60: 479-487; GenBank Accession No. 062760; SEQ ID NO:20305), tiger (GenBank Accession No. 062761; SEQ ID NO:20306), sheep (Arpagaus M, et al., 1991, J. Biol. Chem. 266: 6966-6974; GenBank Accession No. P32753; SEQ ID NO:20607), pig [Arpagaus, 1991 (Supra); GenBank Accession No. P32752; SEQ ID NO:20308], monkey [Arpagaus, 1991 (Supra); GenBank Accession No. P32751; SEQ ID NO:20309], dog [Arpagaus, 1991 (Supra); GenBank Accession No. P32750; SEQ ID NO:20310], bovine [Arpagaus, 1991 (Supra); GenBank Accession No. P32749; SEQ ID NO:20311], rabbit, (Jbilo, O. and Chatonnet, A., 1990, Nucleic Acids Res. 18: 3990; GenBank Accession No. P21927; SEQ ID NO:20312), horse [Moora D R, et al., In: Doctor, B. P., et al., (Eds.); STRUCTURE AND FUNCTION OF CHOLINESTERASES AND RELATED PROTEINS, pp. 145-146, Plenum Press, New York and London (1998); GenBank Accession No. P81908; SEQ ID NO:20313], chicken (GenBank Accession No. NP_{—}989977; SEQ ID NO:20314) sources. Thus, coding sequences information for BChE is available from several databases including the GenBank database available through—www.ncbi.nlm.nih.gov/ and the SwissProt database available through—au.expasy.org/sprot/.

To express exogenous BChE in mammalian cells, a polynucleotide sequence encoding a BChE is preferably ligated into a nucleic acid construct suitable for expression in mammalian cells. Such a nucleic acid construct includes a promoter sequence for directing transcription of the polynucleotide sequence in the cell in a constitutive or inducible manner.

It will be appreciated that the nucleic acid construct of the present invention can utilize BChE as set forth in SEQ ID NO:7 or homologs thereof which exhibit the desired activity (e.g., prevention and/or reversal of amyloid fibril formation). Such homologues can be, for example, at least 70%, preferably, at least 71%, more preferably, at least 72%, more preferably, at least 73%, more preferably, at least 74%, more preferably, at least 75%, more preferably, at least 76%, more preferably, at least 77%, more preferably, at least 78%, more preferably, at least 79%, more preferably, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:7, as determined using the BestFit software of the Wisconsin sequence analysis package, utilizing the Smith and Waterman algorithm, where gap weight equals 50, length weight equals 3, average match equals 10 and average mismatch equals −9.

Similarly, the nucleic acid construct of the present invention includes a polynucleotide encoding a polypeptide at least 70%, preferably, at least 71%, more preferably, at least 72%, more preferably, at least 73%, more preferably, at least 74%, more preferably, at least 75%, more preferably, at least 76%, more preferably, at least 77%, more preferably, at least 78%, more preferably, at least 79%, more preferably, at least 80%, more preferably, at least 81%, more preferably, at least 82%, more preferably, at least 82%, more preferably, at least 83%, more preferably, at least 84%, more preferably, at least 85%, more preferably, at least 86%, more preferably, at least 87%, more preferably, at least 88%, more preferably, at least 89%, more preferably, at least 90%, more preferably, at least 91%, more preferably, at least 92%, more preferably, at least 93%, more preferably, at least 94%, more preferably, at least 95%, more preferably, at least 96%, more preferably, at least 97%, more preferably, at least 98%, more preferably, at least 99% homologous (similar+identical) to the polypeptide set forth by SEQ ID NO:2, as determined using the BlastP software of the National Center of Biotechnology Information (NCBI) using default parameters.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include, for example, the oxidative stress-inducible peroxidase (POD) promoter (Kim K Y, et al., 2003, Plant Mol. Biol. 51: 831-8) which is expected to upregulate the expression BChE in response to the oxidative stress present e.g., in the brain of Alzheimer's patients (Boyd-Kimball D, et al., 2004, Chem. Res. Toxicol. 17: 1743-9), as well as the tetracycline-inducible promoter (Zabala M, et al., 2004, Cancer Res. 64: 2799-804) which can be activated by tetracycline uptake.

It will be appreciated that specific upregulation of BChE expression in amyloid fibril—containing cells or tissues can be achieved using a promoter which is induced in the presence of amyloid fibrils, such as the BACE1 (beta-secretase) (Tong Y, et al., J Neural Transm. 2004 Dec. 22; Epub ahead of print) and caveolin-3 (Nishiyama K, et al., 1999, J. Neurosci. 19: 6538-48) promoters.

The nucleic acid construct (also referred to herein as an “expression vector”) used while implementing the present invention preferably includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the stability (Soreq et al., JMB, 1974) or efficiency of BChE mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech and their derivatives.

To enable secretion of the expressed BChE or BChE derived peptides into the extracellular environment from cells transformed with any of the expression vectors described herein, the expression vector preferably includes additional sequences encoding for signal peptide for seretion being in frame with the sequence encoding for the BChE or BChE derived peptides, so as to allow secretion of the recombinant BChE or derived derived peptides.

Expression vectors containing regulatory elements from eukaryotic viruses such as retroviruses can be also used. SV40 vectors include pSVT7 and pMT2. Vectors derived from bovine papilloma virus include pBV-1MTHA and vectors derived from Epstein Bar virus include pHEBO and p205. Other exemplary vectors include pMSG, pAV009/A⁺, pMTO10/A⁺, pMAMneo-5, baculovirus pDSVE and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of BChE since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

It will be appreciated that increasing the BChE or BChE derived peptide level can be also effected by administration of BChE or BChE derived peptide expressing cells into the individual which cells are capable of secreting BChE or BChE derived peptide into the cellular environment of the amyloid fibrils, i.e., in the tissues where amyloid fibrils are present. Examples for such tissues include, but are not limited to, brain, lung, skin, lymph nodes.

BChE or BChE derived peptide expressing cells can be any suitable cells, such as embryonic stem cells (e.g., embryonic germ cells, embryonic stem cells or cord blood cells), adult stem cells (e.g., bone marrow cells, mesenchymal stem cells, adult tissue stem cells), neuronal cells, hematopoietic cells, keratinocyte cells, lymph node cells which are derived from the individual and are transfected ex vivo with an expression vector containing the polynucleotide designed to express and secrete BCHE or BChE derived peptide as described hereinabove.

Administration of the BChE or BChE derived peptide expressing cells of the present invention can be effected using any suitable route such as intravenous, intra peritoneal, intra spine, intra gastrointestinal track, subcutaneous, transcutaneous, intramuscular, intracutaneous, intrathecal, epidural and rectal. According to presently preferred embodiments, the BChE or BChE derived peptide expressing cells of the present invention are introduced to the individual using intravenous, intra spine and/or intra peritoneal administrations.

BChE or BChE derived peptide expressing cells of the present invention can be derived from either autologous sources such as self bone marrow cells, mesenchymal stem cells and/or adult tissue stem cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60 and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

It will be appreciated that prevention and/or reversing the formation of amyloid fibrils in an individual who is at risk of developing amyloid fibrils (e.g., an individual who is predisposed to an amyloid fibril—related disease or disorder as described hereinbelow) can be effected by transplanting BChE or BChE derived peptide expressing stem cells in the individual. Such cells can be for example, embryonic or adults stem cells [e.g., bone marrow cells, mesenchymal stem cells (MSC)] which following their differentiation in the individual are immune to fibril formation.

It will be appreciated and it is described above in greater detail, increasing the level of BChE and or/BChE derived peptide can also be effected by direct administration of same to a treated individual, preferably formulated in a pharmaceutical composition.

The polynucleotide, polypeptide, peptide or cells expressing and/or secreting same of the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polynucleotide, polypeptide, peptide or cells expressing and/or secreting same accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. The pharmaceutically acceptable carrier can be selected for reducing an immugenicity of the active ingredient, e.g., BChE derived peptide, of the present invention and/or the pharmaceutically acceptable carrier can be designed to allow sustained/controlled and/or slow release of the active ingredient. PEG and liposomes can be used to achieve one or more of these aims.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions and the like, for oral ingestion by a patient. Pharmacological preparations for oral use can be made using a solid excipient, optionally grinding the resulting mixture and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (i.e., the polynucleotide, polypeptide, peptide or cells expressing and/or secreting same) effective to prevent, alleviate or ameliorate symptoms of a disorder (e.g., amyloid fibril—related disease or disorder) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels (e.g., plasma or brain) of the active ingredient are sufficient to prevent amyloid fibril formation (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container and labeled for treatment of an indicated condition, as if further detailed above.

While further reducing the present invention to practice, the present inventors have uncovered that BChE can be used to reduce the acetylcholine—mediated control over inflammatory reactions.

As is shown in Table 8 and described in Example 2 of the Examples section which follows, BCHE, the soluble cholinesterase, is more accessible to circulating ACh than ACHE. In addition, under high concentrations of ACh, BChE's capacity to hydrolyze ACh is only 12-fold lower than that of ACHE. Nevertheless, BCHE constitutes only 10% of the circulation capacity to hydrolyze Ach. Therefore, the present inventors have uncovered that BChE administration shall not increase the inflammatory load, opposite to the case of ACHE administration, which reduces ACh drastically, relieving the blockade over the synthesis by macrophages of pro-inflammatory cytokines (Tracey, 2002). Thus, BChE but not ACHE is predicted to avoid the cholinergic-mediated inflammatory reaction.

Hence, according to an additional aspect of the present invention there is provided a method of limiting or reducing an inflammatory reaction in an individual treated with a cholinesterase. The method according to this aspect of the invention comprises increasing an expression level and/or activity of BChE in the individual, avoiding the risk of inflammatory reaction in the individual. This method may find particular use in treating the inflammatory reactions mediated by circulating organophosphate insecticides or chemical warfare agents, which are oftentimes associated with individuals subjected to occupational or wartime exposure of such agents. To a certain extent, BChE should be viewed as a balancer of the cholinergic status in the peripheral circulation. Because inflammation is conceived as a contributor to numerous neurodegenerative diseases, its capacity to maintain a neutral inflammatory load is an important virtue under conditions requiring prolonged treatment.

Such inflammatory reactions are typically mediated by at least one pro-inflammatory cytokine selected from the group consisting of IL-1, IL-1α, IL-1β, IL-1ss, IL-6, IL-8, IL-10, IL-12, IL-18 and TNFα secreted by cells participating in the inflammatory reactions, e.g., neutrophils, monocytes and eosinophils, or by tissue residing macrophages (Borovikova et al., 2000, Wang et al, 2003).

Increasing the expression level and/or activity of BChE in the individual according to this aspect of the present invention is effected by at least one approach selected from the group consisting of (a) expressing in cells of the individual an exogenous polynucleotide encoding at least a functional portion of BCHE; (b) increasing expression of endogenous BChE in the individual; (c) increasing endogenous BChE activity in the individual; (d) administering an exogenous polypeptide including at least a functional portion of BChE to the individual; and (g) administering BChE expressing cells into the individual. Each one of these approaches is described in greater detail hereinabove.|

The phrase “functional portion” as used herein refers to a part of the BCHE protein (i.e., a polypeptide) which exhibits functional properties of the enzyme such as binding to its substrate. According to preferred embodiments of the present invention the functional portion of BChE is a polypeptide sequence including amino 29-602 (mature BChE protein), optionally, amino acids 1-602 as set forth in SEQ ID NO:2.

Examples of diseases and disorders associated with inflammatory reactions include, but are not limited to, Alzheimer's disease (Nikolov R, 1998, Drug News Perspect. 11: 248-55), sepsis (Wang H, et al., 2004, Nat. Med. 10: 1216-21. Epub 2004 Oct. 24), asthma and chronic obstructive pulmonary disease (COPD) (Gosens R, et al., 2004, Eur. J. Pharmacol. 500: 193-201), rheumatoid arthritis (RA) (Hansel S, et al., 2003, Atherosclerosis. 170: 177-80), Inflammatory bowel disease (e.g., Crohn's disease, ulcerative colitis) (Hatoum O A, et al., 2003, Gastroenterology. 125: 58-69), Sjogren's syndrome (SS) (Borchers A T, et al., 2003, Clin. Rev. Allergy Immunol. 25: 89-104), acute systemic inflammation (Chia S, et al., 2003, J. Am. Coll. Cardiol. 41: 333-9), chronic inflammatory disease, tuberculosis, skin and lung abscesses. All these diseases and disorders can be treated using BChE as an anti-inflammatory agent.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D. and Higgins S. J., Eds. (1985); “Transcription and Translation” Hames, B. D. and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Methods

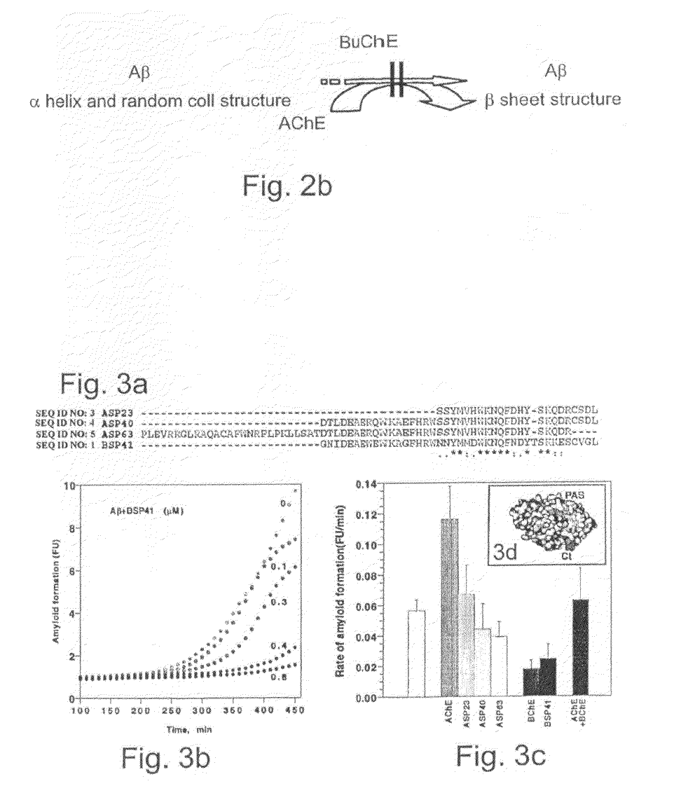

Synthetic peptides—The AChE-S C-terminal (ASP) and the BChE C-terminal (BSP) peptides were synthesized at the noted lengths, using a Pioneer peptide synthesizer (Perspective, Cambridge, UK), purified and analyzed by MALDI-TOFF mass spectrometry as previously described (Grisaru et al., 2001) according to the C-terminal sequences of human ACHE and BCHE, respectively (Glick, 2003). Purity was confirmed by mass spectrometry and was found to be >90%. BSP41 (SEQ ID NO:1) is composed of residues 562-602 in hBChE (SwissProt Accession No. P06276; SEQ ID NO:2). ASP23 (SEQ ID NO:3), ASP40 (SEQ ID NO:4) and ASP63 (SEQ ID NO:5) mimic residues 592-614, 575-614 and 548-610, respectively in hAChE (GenBank Accession No. P22303; SEQ ID NO:6). For peptide sequences and homology see FIG. 3a.

Enzymes—Purified human BChE (from human serun; Sigma, Jerusalem, Israel) and recombinant human ACHE-S, prepared from an ACHE cDNA clone (Soreq et al., 1990) as detailed in Velan et al., 1991 (Sigma, Jerusalem, Israel). Enzyme integrity was verified by measuring the hydrolysis rate of acetyl- or butyrylthiocholine, respectively (Ellman et al., 1961), compared to the protein concentration.

In vitro formation of amyloidfibril—In vitro formation of amyloid fibril was in the presence of the synthetic Aβ (1-40) peptide (Biosource, Camarillo, Calif., USA) as a precursor. The reporter molecule was Thioflavin T (ThT) (Sigma, Cat. No. T-3516, Jerusalem, Israel), a benzothiazole dye that undergoes a shift in its excitation spectrum (from 340 nm to 450 nm) when interacting with β-sheet amyloid structures. The resultant ThT fluorescence signal is proportional to the amount of amyloid formed (LeVine, 1993). A stock solution of Aβ in dimethylsulfoxide (DMSO) was diluted with phosphate-buffered saline (PBS) containing 0.02% Na-Azid to a final concentration of 162 μM and 20 μl of the diluted Aβ solution was placed in each well of a 96 multiwell plate (Nunc, Roskilde, Denmark). After 20 minutes of pre-incubation at room temperature of the Aβ samples (20 μl), 80 μl of 1.25 μM ThT in 50 mM glycine buffer, pH 8.5, was added. Incubation was with shaking at 200 rpm for 6 to 8 hours at the noted temperatures. Fluorescence was measured continuously or at 30-40 minute intervals, using a Spectro-fluorometer (Tecan, Maennedorf, Switzerland). Excitation and emission wavelengths were 450 nm and 485 nm, respectively. Enhancement of the fibrillation process by the different combinations of water-dissolved cholinesterases and peptides mimicking fragments thereof, was evaluated by dissecting the time curves into two stages: the lag before the onset of the fluorescence increase (the nucleation process) and the average rate of fluorescence increase at several time points (rate of fibrils formation). Statistics analysis was performed using the Kaleidagraph Software (Reading, Pa., USA).

Circular Dichroism (CD) measurements—For circular dichroism (CD) measurements, ASP peptides were dissolved in double distilled water (DDW) to a final concentration of 1×10⁻⁴ M. To reach this concentration, as required for the CD measurements, BSP had to be dissolved in 1,1,1,3,3,3-hexafluoro-2-propanol (HFIP). Direct CD spectra were recorded at room temperature using a CD Jasco J-810 Spectropolarimeter (Easton, Md., USA) with a 1 mm path length cell. Recordings were at 0.5 nm intervals in the spectral range 185-260 nm.

Peptide modeling—Peptide modeling involved virtual construction of the analyzed peptides using the interface of the Deep View spdbv 3.7 software (Glaxo Smith Kline, Bredford, UK) followed by distance geometry minimization. Figures were created with the PyMol software (DeLano Scientific LLS, San Carlos, Calif., USA). Helical Wheel Projections were done by wheel. PI, Version 0.10 (Cell Biology and Neuroscience, UC Riverside, Calif., USA).

BChE biochemistry—Serum cholinesterase catalytic activity measurements are based on a spectrophotometric method adapted to a microtiter plate assay. Butyrylthiocholine (BTCh, Sigma) hydrolysis rates are measured following 20 minutes incubation with 5×10⁻⁵ M tetraisopropyl pyrophosphoramide (iso-OMPA, Sigma), a specific BChE inhibitor or 10⁻⁵ M 1,5-bis(4-allyldimethylammoniumphenyl) pentan-30-one dibromide (BW284c51, Sigma, A9013), a specific AChE inhibitor. Addition of both inhibitors reduces hydrolysis to the rate of spontaneous hydrolysis measured in control reactions lacking enzyme or substrate, attesting to the specificity of these serum activities. Readings at 405 nm are repeated at 2-minute intervals for 20 minutes. Non-enzymatic hydrolysis of substrate is subtracted from the total rate of hydrolysis. Enzyme activity is calculated using the molar extinction coefficient for 5-thio-2-nitrobenzoate [13,600 M⁻¹×cm⁻¹] [Ellman, G. L. et al. (1961) Biochem. Pharmacol. 7:88-95].

MPTP poisoning of mice—After its accidental discovery in the early 1980s, 1-methyl-4-phenyl-1,2,3,6-tetrahydropyridine (MPTP) has been shown to induce Parkinsonism in monkeys and Parkinson-like symptoms in mice, both at the behavioral and the anatomical level. Thus, MPTP has been used extensively as a model for Parkinson's disease in non-human primates and mice (Predborzski et al. (2000) Restorative Neurology and Neuroscience 16:135-142). To induce Parkinson-like symptoms each mouse is injected with a 0.01 ml of an MPTP solution (2 mg/ml) per gram mouse weight (e.g. a 20 gram mouse receives 0.2 ml).

Telemetric follow-up of behavior—Battery operated biotelemetric transmitters (model VM-FH, Mini Mitter, Sun River, Oreg., USA) are implanted in the peritoneal cavity under ether anesthesia 12 days prior to the test. After implantation, mice are housed in separate cages with free access to food and water. Output is monitored by a receiver board (model RA-1010, Mini Mitter) placed under each animal's cage and fed into a peripheral processor (BCM 100) connected to a desktop computer. Locomotor activity after the dark/light shift is measured by detecting changes in signal strength as animals move about in their cages, so that the number of pulses generated by the transmitter is proportional to the distance the animal moves. The cumulative number of pulses generated over the noted periods is recorded [Yirmiya, R. et al. (1997) Brain Res. 749: 71-81]. Recording lasts 24 consecutive hours, starting at 9:30 am, with the light phase of a 12:12 hour dark/light cycle beginning at 7:00 am. To initiate a day/night switch, the dark/light periods are reversed and recording starts 72 hours after the switch and lasts 24 hours. Recording proceeds for an additional 3 hours after injection of active agents.

### Example 1

### BChE Inhibits Aβ Fibril Formation

The progressive deposition of amyloid β peptide (Aβ) in fibrillar form is a key feature in the development of the pathology in Alzheimer's disease. ACHE is found associated with amyloid plaque deposits [Urlich, J. et al. (1990) Acta Neuropathol. (Berl.) 80(6):624-8]. In addition, in vitro studies demonstrated that ACHE promotes the assembly of Aβ peptide into amyloid fibrils [Alvarez et al. (1995) Neurosci. Lett. 201(1): 49-52; Inestrosa et al. (1996a); Inestrosa et al. (1996b); Alvarez et al. (1998)]. The fluorogenic incorporation of Thioflavin T into Aβ fibrils essentially measures the shift from an amorphic, unstructured polypeptide into a tight network of β-pleated sheets which initiates the formation of the amyloid plaques.

In vitro Aβ fibrils form spontaneously, provided that the peptide is present at the certain critical concentration. At undisturbed conditions the fibrils are formed in the time span of days (2-7 days) but this process can be considerably accelerated by shaking the solution. The fibril formation can be followed by the measurements of: (1) turbidity of the solution, (2) staining with diazobenzidine sulfonate dye, Congo Red, or by (3) staining with benzothiazole dye, Thioflavin T (ThT). The latter may be added at the end of the procedure, or, alternatively, at the beginning in which case it provides a real-time follow-up of fibril formation.

The inventors were able to follow the Aβ fibrils generation, in vitro, using all the above mentioned methods, with a preference for real-time ThT fluorescence measurements, as the most sensitive and reproducible method.

Experimental Results

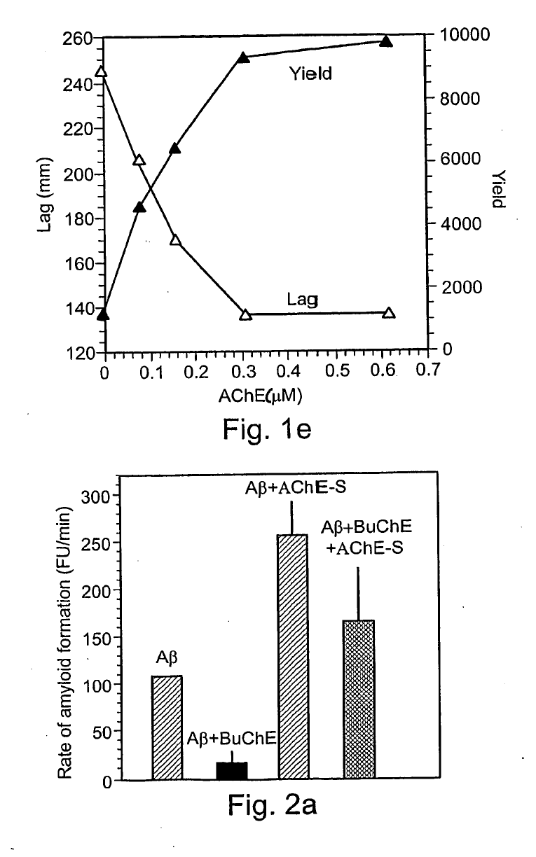

BChE attenuates amyloid fibrils formation—Amyloid fibril (Aβ) formation was quantified by measuring changes in ThT fluorescence. As predicted, Aβ fibrils were spontaneously formed in vitro provided that the Aβ peptide was present above 5 μM. FIGS. 1a-e present the outcome of characteristic experiments, demonstrating the kinetics of amyloid β sheets formation from Aβ [1-40] peptide at a concentration of 33 μM. Reactions were performed in the absence or presence of increasing concentrations of purified human BChE (SEQ ID NO:2; FIG. 1a), recombinant ACHE-S (SEQ ID NO:6; FIG. 1b) or both (FIGS. 1c and d). At the general range of 1:100 ratio of BChE to Aβ, purified BChE surprisingly prolonged the lag and reduced the apparent rate of amyloid formation. Due to the complex kinetics of the fibril formation process the apparent rate of change in ThT fluorescence was repeatedly determined during the exponential phase of increase in its signal (at 390±20 minutes). This measurement resulted in a dose-dependent suppression pattern, to the extent that addition of 0.4 μM BChE to 33 μM Aβ totally prevented fibril formation for over 600 minutes (FIG. 1a). In contrast, addition of similar doses of AChE to Aβ, predictably shortened by half the lag time prior to fibril formation, again in a dose dependent manner (FIG. 1e). The lag period therefore decreased from 240 minutes to 150 minutes and the maximal rate of fibril formation increased from 0.057 to 0.116 fluorescence units (FU)/min for Aβ alone as compared to Aβ in the presence of 0.36 μM AChE-S (FIG. 1b and Table 7, hereinbelow). When increasing doses of hBChE were added to a combined mix of 33 μM Aβ with 0.36 μM AChE-S, a dose-dependent interference with the fibril formation process was observed (FIG. 1c and 2a). Thus, BChE is capable of reducing the rate of fibrillation of Aβ alone, or Aβ which is formed in the presence of AChE.

TABLE 7
Reproducibility and significance of the modified fibrillation effects

| | | | | | Rate | | |
|---|---|---|---|---|---|---|---|
| | | Concentration | | Rate | (fold | Lag | Lag (fold |
| | n | (mg/L) | M.W. | (FU/h) | change) | (h) | change) |
| 1. No addition | 22 | — | — | 3.4 ± 0.4 | — | 4.0 ± 0.3 | — |
| 2. AChE-S | 16 | 25.8 | 64575.1 | 7.0 ± 1.3 | 2.1* | 2.5 ± 0.3 | 0.6* |
| 3. ASP23 | 9 | 1.2 | 2875.1 | 4.0 ± 1.1 | 1.2 | 4.5 ± 0.7 | 1.1 |
| 4. ASP40 | 8 | 2.0 | 5074.5 | 2.7 ± 1.0 | 0.8 | 4.4 ± 0.5 | 1.1 |
| 5. ASP63 | 8 | 3.1 | 7752.7 | 2.4 ± 0.6 | 0.7 | 5.9 ± 0.9 | 1.5 |
| 6. BChE | 12 | 27.4 | 68418.1 | 1.1 ± 0.3 | 0.3* | 5.6 ± 0.5 | 1.4* |
| 7. BSP41 | 6 | 2.0 | 5029.5 | 1.5 ± 0.6 | 0.4* | >7.5 | >1.9* |

Table 7: Aβ fibrillation was characterized as detailed under Methods, for the noted No. of repetitions (n) in each case. Lag and rate values are expressed as mean ± Standard Error (SE). Statistically significant differences from control (No addition) are marked by asterisks. h = hours; Note the different time scale (h).

Altogether, as is schematically illustrated in FIG. 2b, BChE exhibits an inhibitory effect on amyloid fibril formation and thus counteracts the acceleration effect formed by ACHE.

BSP attenuates fibril formation—In an attempt to identify the region(s) within the BChE molecule which are responsible for interfering with Aβ fibrillation, homologous peptides corresponding to the C-terminal domains of BChE and AChE-S were synthesized and their effect on amyloid fibrils formation was examined. FIG. 3a presents the analyzed sequences and demonstrates the significant homology between them. As is shown in FIGS. 3b and c, the 41-amino acid long BSP41 peptide (SEQ ID NO:1) was capable of interfering with Aβ fibrillation in a dose dependent manner and in similar molar ratios as with BChE (FIG. 3b-c). Moreover, as is seen in Table 7 hereinabove, absolute dose calculations revealed that the BSP41 peptide was even more potent in interfering with Aβ fibrillation than the complete BChE enzyme since only 2 μg/ml BSP peptide were needed for a complete attenuation of Aβ fibrillation (for 400 minutes) as compared with 30 μg/ml of the complete BChE enzyme.

ASP peptides do not activate or inhibit Aβ fibrillation—In contrast to BSP41, the 40-amino acid long ASP40 peptide (SEQ ID NO:4) mimicking the corresponding domain in AChE-S failed to show any significant capacity to activate or inhibit fibrillation (FIG. 3c). Similar experiments utilizing the two additional peptides as putative modifiers of Aβ fibril formation: ASP63 (SEQ ID NO:5), a longer version of the ACHE-S C-terminus and ASP23 (SEQ ID NO:3), a shorter 23-amino acid long ACHE-S C-terminus peptide, resulted in no significant effect on amyloid formation (FIG. 3c). These results demonstrate that the entire C-terminal domain in AChE-S is unlikely involved in the Aβ fibrillation process; serving as a negative control, these findings further provide a proof of the specificity of the PSP41 effect.

It is worth mentioning, that in both ACHE and BChE, the C-terminus is positioned far away from the Peripheral Anionic binding Site (PAS) domain which is considered by many investigators to be causally involved in Aβ fibrillation (De Ferrari G V, et al., 2001, Biochemistry. 40: 10447-57). FIG. 3d presents the cholinesterase structure, demonstrating these physical distances.

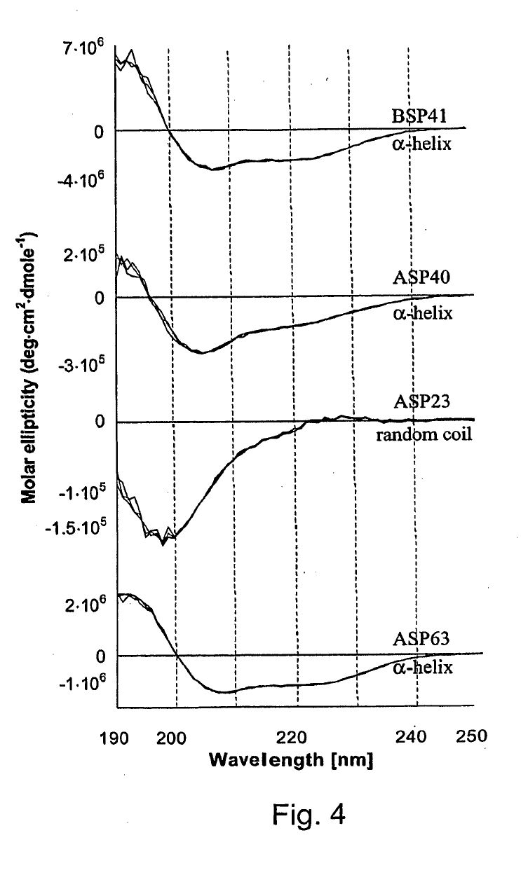

Molecular modeling points at putative structural differences between BSP and ASP—In search for structural differences between the BSP and ASP peptides, the circular dichroism (CD) properties of the synthesized series of BSP and ASP peptides were determined (FIG. 4). Due to difficulties to solubilize BSP in water up to the concentrations required for CD tests, HFIP was used as a solvent. Molar elipticity measurements for BSP41, ASP40 and ASP63 all showed a clear positive band at 192 nm and a negative band at 209 nm, characteristic of a helical structures. The helical features observed for ASP40 are in line with reports of others (Cottingham et al., 2003). ASP23 showed, however, a clear negative band at 195 nm, characteristic of a random coil structure.

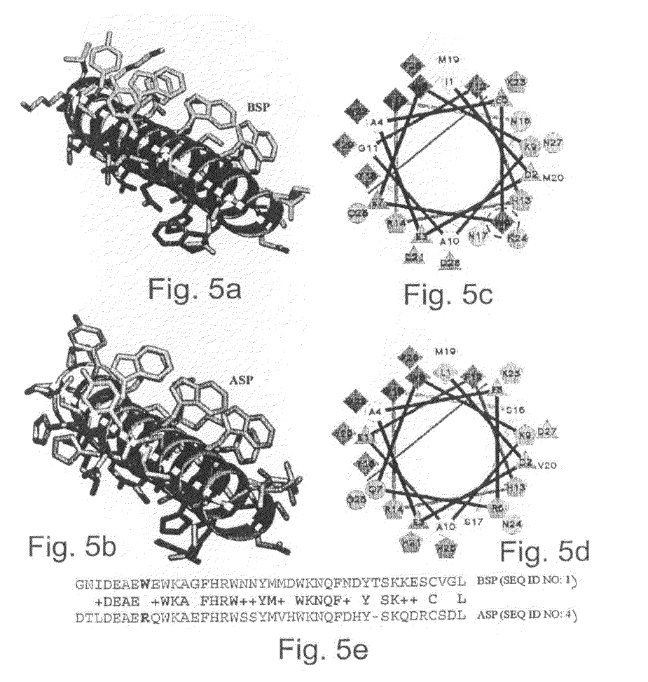

To further pursue the structural basis for the functional differences between ASP and BSP, the structures of the ASP40 and BSP41 peptides were modeled (FIGS. 5a-e). Both peptides emerged as symmetric amphipathic helices with similar distributions of polar and non-polar residues. However, BSP's amphipathicity appeared to be locally disturbed by a protruding aromatic trytophane residue in the polar side of the helix. Further studies will be required to find out if this local structural difference between the ASP and BSP peptides is the cause of asymmetry and functional differences.

Altogether these results demonstrate that BChE, in a molar ratio of 1:100 to the Aβ peptide, is efficiently capable of slowing down the fibrillogenic process; BChE was found to retard the onset of fluorogenic increase and reduce the rate of that increase, once initiated. Moreover, when added to Aβ together with ACHE, BChE is capable of delaying the onset and reducing the rate of fibril formation in a dose-dependent manner. In addition, BSP, a peptide mimicking the C-terminus of BChE, was found to be highly potent in inhibiting Aβ fibril formation. Thus, the BSP peptide, at a concentration as low as 2 μg/ml was capable of suppressing Aβ fibril formation for as long as 400 minutes, similar to the effect obtained using 30 μg/ml of the complete BChE enzyme (Table 7, hereinabove).

These results suggest the use of BChE and/or peptide derived thereof as novel therapeutic agents useful for treating diseases associated with fibrils formation and deposition such as Alzheimer's disease (AD).

Discussion

By following amyloid formation with time (rate determination), rather than measuring the final yield of the fibrillation process the present inventors were able to show that BChE prolongs the lag and reduces the rate of amyloid fibrils formation in vitro. Others observed that BChE does not enhance the fibrillation process, but interpreted that to imply no involvement (Inestrosa et al., 1996b). The present study, however, demonstrated that contrary to this early prediction, BChE acts as a negative modifier in this process and that it likely does that through the action of its C-terminal peptide, BSP.

The capacity of BChE and BSP to suppress amyloid fibril formation was observed both at the nucleation and the progression phases of the fibrillation process and was dose-dependent, a mirror image of the facilitation observed with recombinant, highly purified hAChE-S. Importantly, a corresponding series of peptides designed to mimic increasingly long C-terminal regions of AChE-S failed to affect the fibrillation process and showed neither facilitation nor suppression of amyloid fibrils formation. This, in turn, suggests that the fibrillation enhancement conferred by AChE-S does not involve the C-terminal domain of this enzyme, compatible with suggestions that it is induced by the peripheral anionic site (PAS,) (Inestrosa et al., 1996a).

BSP41, the 41-amino acids long peptide representing the C-terminus of BChE, showed solubility differences when compared to ASP40. BSP, but not ASP, further induced effective suppression of the fibrillation process, as potent as that of the inhibitory effect of intact BChE. This supports the hypothesis that BSP by itself is the cause for BChE's modifying effect of the Aβ fibril formation process. The secondary structure of the synthetic peptides employed and which mimic the C-terminal peptides of AChE-S and BChE, obviously depends on the length of the specific peptide tested. The ASP63 and ASP40 residue peptides are α-helical, whereas the shorter ASP23 shows a random coil structure. However, none of the ASP23, ASP40, ASP63 peptides could by themselves facilitate Aβ fibrillation. That ASP shares some sequence homology with the Aβ peptide (Cottingham et al., 2002; Greenfield and Vaux, 2002) thus appears irrelevant to the phenomenon observed in the present study. Thus, without being bound with any theory it is likely that AChE-S promotes fibrillation via its PAS domain (Inestrosa et al., 1996b), whereas BChE's C-terminal peptide actively interferes with this process, most likely, by heterologous hydrophobic interaction.

The solubility differences, CD properties and structural features observed for BSP and ASP are all compatible with the hypothesis that the difference in the effects may reside in their distinct amphipatic characteristics. While the ASP40 peptide shows a clear division between hydrophobic and hydrophilic moieties, in BSP such a clear division appears impossible. Site-directed mutagenesis may be used to test if the hydrophobicity properties of BSP enable binding to AD pre-fibrils and control the attenuation of the fibrillation process.

The BChE K variant, representing a single C-terminal substitution (A539T) shows 30% reduction in hydrolytic activity (Bartels et al., 1992). This allele occurs with relatively high incidence (12% of the Caucasian population), which allowed testing its incidence in AD patients. Intriguingly, some (Lehmann et al., 1997; Lehmann et al., 2000) but not others (Brindle et al., 1998) reported association of this variant with an increased risk of late-onset AD. While this increased risk was tentatively attributed to the reduced hydrolytic activity of the K variant, the possibility of Aβ fibrillation effect should be explored.

The AChE-S protein facilitates Aβ fibrillation both in vitro and in vivo (Inestrosa et al., 1996a; Inestrosa et al., 1996b; Munoz and Inestrosa, 1999). Based on this precedence, it is tempting to speculate that BChE and BSP would also affect the Aβ fibrillation process in vivo, suggesting that the ratio between AChE-S and BChE may affect plaque formation. In the human brain, ACHE mRNA is 20-fold more abundant than BChE mRNA (Soreq and Zakut, 1993). In human blood, however, BChE, at 50 nM is 3-fold more abundant than ACHE (Loewenstein-Lichtenstein et al., 1995). The present findings imply that this concentration is sub-optimal for attenuating Aβ fibrils formation. This, in turn, suggests a therapeutic use of BSP, a relatively short mimic of the C-terminal peptide of BCHE. BSP can by itself attenuate Aβ fibrillation in the low dose of 2 mg/L. In view of the theory that the Aβ fibrillation process involves continuous communication between the brain and the circulation (Basun et al., 2002), administration of BSP may be by injection, similar to erythropoietin or GM-CSF (Arndt et al., 2004; Zhang et al., 2005). An alternative option for BSP administration may be by constructing a BSP expression vector and using this vector for transfecting bone marrow cells for autologous transplantation, similar to the gene therapy protocols used for adenosine deaminase replacement (Aiuti et al., 2003; Herzog and Arruda, 2003). In either way, the disrupted blood-brain barrier of AD patients (see Soreq, 2002 for a recent review) predicts effective penetrance of this peptide into the brain. The role(s) and actions of BChE in the pathogenesis of Alzheimer's disease thus merit renewed attention.

### Example 2

### BChE Can be Used to Limit ACH-Mediated Inflammatory Response

In normal human serum, an average assay of 20 individuals yields 81±23 nmol butyrylthiocholine (BThCh) hydrolyzed/hr/μl serum (assayed at 2 mM BThCh). Out of the total ACh binding sites in the human blood, BChE provides 75% or 50 nM [Lowenstein et al. (1995) Mol. Cell. Biol. Hum. Dis. Ser. 5:307-49] and erythrocytes ACHE—25%, or 10 nM [Ott et al. (1982) FEBS Lett. 138(2): 187-9]. However, the capacity of ACHE to degrade ACh decreases above 3 mM (which is defined as “substrate inhibition”). In contrast, BChE's capacity to hydrolyze ACh increases under increased ACh concentration (“substrate activation”). Therefore, conditions of elevated ACh (i.e. acute stress injury or exposure to anticholinesterases both of which increase the risk of cognitive decline), should best be treated with BChE, because under such conditions AChE's hydrolytic activity will be impaired but BChE's will be facilitated.

An inflammatory reaction, for example, as a response to an injury, involves the production of pro-inflammatory cytokines (e.g., by tissue macrophages). Intriguingly, the neurotransmitter which controls such a process is acetylcholine (ACh) [Bernik, T. R. et al. (2002) J. Exp. Med. 195(6):781-8]. In both tissues and circulation, ACh levels are controlled by ACHE [Soreq, H. and Seidman, S. (2001) Nature Neurosci. Rev. 2:294-302]. Therefore, increased AChE can initiate inflammatory reactions because it reduces ACh levels and increases production of cytokines. However, while inflammatory reactions are apparently useful in the short range, they carry a significant long-range risk of neurodegenerative disease. For example, head injury induces the largest non-inherited risk of AD [Shohaini, E. et al. (2000) J. Mol. Med. 78:228-236]. Therefore, both therapeutic uses of recombinant ACHE and treating patients with anti-cholinesterases, which induce ACHE overproduction as a feedback response, carry an inherent risk of increasing the inflammatory load in treated patients.

Unlike AChE, BChE does not entail a risk of increasing the inflammatory load, since it is not part of the auto-regulatory feedback loop of injury-cytokine release-cholinergic imbalance. In addition, BChE is soluble and thus accessible to circulating ACh. Moreover, the substrate of preference of BChE is different than that of ACHE. Thus, over the range of substrate concentrations 0.1 to 25 mM, the ratio of hydrolysis of AThCh relative to BThCh differs between the enzymes (Table 8, hereinbelow).

TABLE 8
Rate of hydrolysis of AThCh/BThCh*

| Substrate (mM) | 0.1 | 25.0 |
|---|---|---|
| hSerum BChE | 0.45 | 0.20 |
| hRBC AChE | 120 | 7.2 |

Table 8: The ratio of hydrolysis of AThCh relative BThCh is presented for two substrate concentrations (0.1 and 25 mM). The results present the average of three experiments.

Thus, under normal condition and assuming low ACh levels and negligible soluble ACHE levels in the circulation, the total ACh hydrolyzing capacity will be divided as follows: 75% by BChE and 25% by ACHE, when the ratio is 3:1.

Based on the AThCh hydrolysis ratio differences of AChE:BChE [245-fold at 0.1 mM, Table 8, hereinabove], BChE capacity of ACh hydrolysis in the circulation will be ca. 80-fold lower than that of ACHE under normal circumstances. However, increased ACh levels (up to 10 mM, as is the concentration in synapses) will improve BChE capacity to hydrolyze ACh (as in 25 mM, the difference is 36-fold, or only 12-fold lower than ACHE, Table 8, hereinabove). Nevertheless, BChE administration shall not increase the inflammatory load, because even at these higher ACh concentrations BChE will only constitute 10% of the circulation capacity to hydrolyze ACh. The progressive age-dependent increase in inflammatory diseases should hence be attributed to the increase in circulation ACHE, not to BCHE. In addition, mice injected with paraoxon, the OP metabolite of parathion, show reduced locomotion and decreased body temperature (Coudray-Lucas C, et al., 1983, Acta Pharmacol Toxicol (Copenh). 52: 224-9; Beeri R, et al., 1995, Curr. Biol. 5: 1063-71).

Based on the above discussion, the present inventors have uncovered that injection of human recombinant BChE (hrBChE) will limit the inflammatory reaction and reduce production of pro-inflammatory cytokines. Thus, the present inventors suggest the use of BChE in preventing and/or limiting ACh-modulated inflammatory reactions. The prediction is that treatment with BChE will reduce ACh levels below the threshold inducing ACHE overproduction (as in Kaufer et al., 1998, Meshorer et al., 2002), thus avoiding the relief over macrophages capacity to produce pro-inflammatory cytokines.

### Example 3

### BChE Inhibits Amylin Fibrillation

Type II diabetes is associated with widespread amyloidosis of the pancreatic islet β-cell (for review see Hoppener J W, et al., 2000, N. Engl. J. Med. 343(6): 411-9). Although the presence of amyloid deposits in diabetes was first recorded over 100 years ago, the contribution to the pathogenesis of diabetes is only now being appreciated (Hoppener J W, et al., 2002, Mol. Cell. Endocrinol. 197(1-2): 205-12). The polypeptide core of these deposits has been independently identified as islet amyloid polypeptide (IAPP) or amylin. Recent work has shown that amylin amyloid formation is cytotoxic and diabetogenic [Hoppener, 2002 (Supra)].

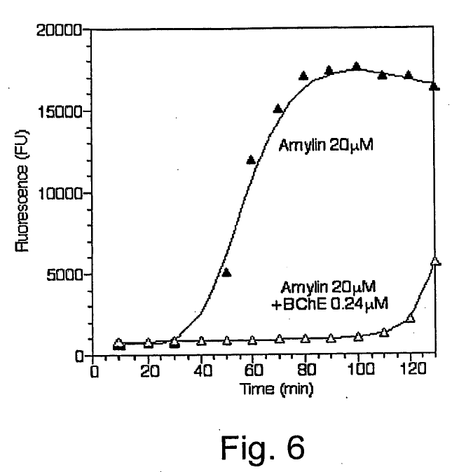

To test the ability of BChE to prevent amylin amyloid fibril formation, the present inventors have tested the rate of amylin fibrillation (at a concentration of 20 μM) in the presence or absence of 0.24 μM BChE (purified from pooled human serum). As is shown in FIG. 6, BChE completely attenuated the formation of amylin fibrils for 100 minutes, and increased the lag time of amyloid fibril formation from 30 minutes to 110 minutes.

These results demonstrate the ability of BChE to inhibit amylin fibril formation and suggest the use of BChE as a therapeutic agent for the prevention of amylin amyloidosis and the treatment of type II diabetes.

To further test the capacity of BChE to prevent amylin amyloidosis in vivo, BChE can be administered into transgenic mice over-expressing huIAPP (amylin) in pancreatic islet β-cells (Soeller W C, et al., 1998, Diabetes, 47(5): 743-50) and the effect of BChE in prevention or reversal of amylin amyloidosis can be determined using histopathological and immunostaining analyses.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES LISTED IN ALPHABETIC ORDER

### Additional References are Cited in the Text

1. Aiuti, A., Ficara, F., Cattaneo, F., Bordignon, C. and Roncarolo, M. G. (2003) Gene therapy for adenosine deaminase deficiency. Curr Opin Allergy Clin Immunol 3: 461-466.
2. Alvarez, A., Alarcon, R., Opazo, C., Campos, E. O., Munoz, F. J., Calderon, F. H., Dajas, F., Gentry, M. K., Doctor, B. P., De Mello, F. G. and Inestrosa, N. C. (1998) Stable complexes involving acetylcholinesterase and amyloid-beta peptide change the biochemical properties of the enzyme and increase the neurotoxicity of Alzheimer's fibrils. J Neurosci 18: 3213-3223.
3. Arndt, U., Kaltwasser, J. P., Gottschalk, R., Hoelzer, D. and Moller, B. (2004) Correction of iron-deficient erythropoiesis in the treatment of anemia of chronic disease with recombinant human erythropoietin. Ann Hematol.
4. Bartels, C. F., Jensen, F. S., Lockridge, O., van der Spek, A. F., Rubinstein, H. M., Lubrano, T. and La Du, B. N. (1992) DNA mutation associated with the human butyrylcholinesterase K-variant and its linkage to the atypical variant mutation and other polymorphic sites. Am J Hum Genet. 50: 1086-1103.
5. Bartolini, M., Bertucci, C., Cavrini, V. and Andrisano, V. (2003) beta-Amyloid aggregation induced by human acetylcholinesterase: inhibition studies. Biochem Pharmacol 65: 407-416.
6. Basun, H., Nilsberth, C., Eckman, C., Lannfelt, L. and Younkin, S. (2002) Plasma levels of Abeta42 and Abeta40 in Alzheimer patients during treatment with the acetylcholinesterase inhibitor tacrine. Dement Geriatr Cogn Disord 14: 156-160.
7. Borovikova, L. V., Ivanova, S., Zhang, M., Yang, H., Botchkina, G. I., Watkins, L. R., Wang, H., Abumrad, N., Eaton, J. W., and Tracey, K. J. (2000) Vagus nerve stimulation attenuates the systemic inflammatory response to endotoxin. Nature 405: 458-462.
8. Brindle, N., Song, Y., Rogaeva, E., Premkumar, S., Levesque, G., Yu, G., Ikeda, M., Nishimura, M., Paterson, A., Sorbi, S., Duara, R., Farrer, L. and St George-Hyslop, P. (1998) Analysis of the butyrylcholinesterase gene and nearby chromosome 3 markers in Alzheimer disease. Hum Mol Genet. 7: 933-935.
9. Cottingham, M. G., Hollinshead, M. S. and Vaux, D. J. (2002) Amyloid fibril formation by a synthetic peptide from a region of human acetylcholinesterase that is homologous to the Alzheimer's amyloid-beta peptide. Biochemistry 41: 13539-13547.
10. Cottingham, M. G., Voskuil, J. L. and Vaux, D. J. (2003) The intact human acetylcholinesterase C-terminal oligomerization domain is alpha-helical in situ and in isolation, but a shorter fragment forms beta-sheet-rich amyloid fibrils and protofibrillar oligomers. Biochemistry 42: 10863-10873.
11. Darvesh, S., Hopkins, D. A. and Geula, C. (2003) Neurobiology of butyrylcholinesterase. Nat Rev Neurosci 4: 131-138.
12. De Ferrari, G. V., Mallender, W. D., Inestrosa, N. C. and Rosenberry, T. L. (2001) Thioflavin T is a fluorescent probe of the acetylcholinesterase peripheral site that reveals conformational interactions between the peripheral and acylation sites. J Biol Chem 276: 23282-23287.
13. Ellman, G. L., Courtney, D. andres, V. J. and Featherstone, R. M. (1961) A new and rapid colorimetric determination of acetylcholinesterase activity. Biochem. Pharmacol. 7: 88-95.
14. Geula, C. and Mesulam, M. M. (1995) Cholinesterases and the pathology of Alzheimer disease. Alzheimer Dis Assoc Disord 9 Suppl 2: 23-28.
15. Glick, D., Ben Moyal, L. and Soreq, H. (2003) Genetic variation in butyrylcholinesterase and the physiological consequences for acetylcholinesterase function. London: Martin Dunitz.
16. Greenfield, S, and Vaux, D. J. (2002) Parkinson's disease, Alzheimer's disease and motor neurone disease: identifying a common mechanism. Neuroscience 113: 485-492.
17. Grisaru, D., Deutsch, V., Shapira, M., Pick, M., Stemfeld, M., Melamed-Book, N., Kaufer, D., Galyam, N., Gait, M. J., Owen, D., Lessing, J. B., Eldor, A. and Soreq, H. (2001) ARP, a peptide derived from the stress-associated acetylcholinesterase variant, has hematopoietic growth promoting activities. Mol Med 7: 93-105.
18. Herzog, R. W. and Arruda, V. R. (2003) Update on gene therapy for hereditary hematological disorders. Expert Rev Cardiovasc Ther 1: 215-232.
19. Inestrosa, N. C., Alvarez, A. and Calderon, F. (1996a) Acetylcholinesterase is a senile plaque component that promotes assembly of amyloid beta-peptide into Alzheimer's filaments. Mol Psychiatry 1: 359-361.
20. Inestrosa, N. C., Alvarez, A., Perez, C. A., Moreno, R. D., Vicente, M., Linker, C., Casanueva, O. I., Soto, C. and Gamido, J. (1996b) Acetylcholinesterase accelerates assembly of amyloid-beta-peptides into Alzheimer's fibrils: possible role of the peripheral site of the enzyme. Neuron 16: 881-891.
21. Kaufer, D., Friedman, A., Seidman, S, and Soreq, H. (1998). Acute stress facilitates long-lasting changes in cholinergic gene expression. Nature 393, 373-377.
22. Lambert, M. P., Barlow, A. K., Chromy, B. A., Edwards, C., Freed, R., Liosatos, M., Morgan, T. E., Rozovsky, I., Trommer, B., Viola, K. L., Wals, P., Zhang, C., Finch, C. E., Krafft, G. A. and Klein, W. L. (1998) Diffusible, nonfibrillar ligands derived from Abetal-42 are potent central nervous system neurotoxins. Proc Natl Acad Sci USA 95: 6448-6453.
23. Lehmann, D. J., Johnston, C. and Smith, A. D. (1997) Synergy between the genes for butyrylcholinesterase K variant and apolipoprotein E4 in late-onset confirmed Alzheimer's disease. Hum Mol Genet. 6: 1933-1936.
24. Lehmann, D. J., Nagy, Z., Litchfield, S., Borja, M. C. and Smith, A. D. (2000) Association of butyrylcholinesterase K variant with cholinesterase-positive neuritic plaques in the temporal cortex in late-onset Alzheimer's disease. Hum Genet. 106: 447-452.
25. LeVine, H., 3rd (1993) Thioflavine T interaction with synthetic Alzheimer's disease beta-amyloid peptides: detection of amyloid aggregation in solution. Protein Sci 2: 404-410.
26. Loewenstein-Lichtenstein, Y., Schwarz, M., Glick, D., Norgaard Pedersen, B., Zakut, H. and Soreq, H. (1995) Genetic predisposition to adverse consequences of anti-cholinesterases in ‘atypical’ BChE carriers. Nature Med 1: 1082-1085.
27. Meshorer, E., Erb, C. Gazit, R., Pavlovsky, L., Kaufer, D., Glick, D., Friedman, A., Ben-Arie, N. and Soreq, H. (2002). Alternative splicing and neuritic mRNA translocation under long-term neuronal hypersensitivity. Science, 295, 508-512.
28. Mesulam, M., Carson, K., Price, B. and Geula, C. (1992) Cholinesterases in the amyloid angiopathy of Alzheimer's disease. Ann Neurol 31: 565-569.
29. Mesulam, M. M. and Geula, C. (1994) Butyrylcholinesterase reactivity differentiates the amyloid plaques of aging from those of dementia. Ann Neurol 36: 722-727.
30. Mesulam, M. M., Guillozet, A., Shaw, P., Levey, A., Duysen, E. G. and Lockridge, O. (2002) Acetylcholinesterase knockouts establish central cholinergic pathways and can use butyrylcholinesterase to hydrolyze acetylcholine. Neuroscience 110: 627-639.
31. Morel, N., Leroy, J., Ayon, A., Massoulie, J. and Bon, S. (2001) Acetylcholinesterase H and T dimers are associated through the same contact. Mutations at this interface interfere with the C-terminal T peptide, inducing degradation rather than secretion. J Biol Chem 276: 37379-37389.
32. Munoz, F. J. and Inestrosa, N. C. (1999) Neurotoxicity of acetylcholinesterase amyloid beta-peptide aggregates is dependent on the type of Abeta peptide and the ACHE concentration present in the complexes. FEBS Lett 450: 205-209.
33. Prody, C. A., Zevin-Sonkin, D., Gnatt, A., Goldberg, I. and Soreq, H. (1987) Isolation and characterization of full-length cDNA clones coding for cholinesterase from fetal human tissues. Proc. Natl. Acad. Sci. USA 84, 3555-3559.
34. Rees, T., Hammond, P. I., Soreq, H., Younkin, S, and Brimijoin, S. (2003) Acetylcholinesterase promotes beta-amyloid plaques in cerebral cortex. Neurobiol Aging 24: 777-787.
35. Rees, T. M. and Brimijoin, S. (2003) The role of acetylcholinesterase in the pathogenesis of Alzheimer's disease. Drugs Today (Barc) 39: 75-83.
36. Rees, T. M., Berson, A., Sklan, E. H, Younkin, L., Younkin, S., Brimijoin and Soreq, H. (2005 in press) Memory deficits correlating with acetylcholinesterase splice shift and amyloid burden in doubly transgenic mice. Current Alzheimer Research.
37. Selkoe, D. J. (1996) Amyloid beta-protein and the genetics of Alzheimer's disease. J Biol Chem 271: 18295-18298.
38. Soreq, H., Ben-Aziz, R., Prody, C. A., Seidman, S., Gnatt, A., Neville, L., Lieman-Hurwitz, J., Lev-Lehman, E., Ginzberg, D., Lipidot-Lifson, Y. and Zakut, H. (1990) Molecular cloning and construction of the coding region for human acetylcholinesterase reveals a G+C-rich attenuating structure. Proc Natl Acad Sci USA 87: 9688-9692.
39. Soreq, H. and Zakut, H. (1990) Amplification of butyrylcholinesterase and acetylcholinesterase genes in normal and tumor tissues: putative relationship to organophosphorous poisoning. Pharm Res 7: 1-7.
40. Soreq, H., Kaufer, Daniela Glick, David Friedman, Alon (2002) The molecular biology of Blood Brain Barrier disruption under stress. In Brain Disease: Therapeutic Strategies and Repair. O. Abramsky, D. A., A Miller, G. Said (ed): Martin Dunitz, pp. 231-238.
41. Soreq, H., Nudel, U., Salomon, R., Revel, M. and Littauer, U. Z. (1974) In vitro translation of polyadenylic acid-free rabbit globin messenger RNA. J. Mol. Biol. 88, 233-245.
42. Sponne, I., Fifre, A., Drouet, B., Klein, C., Koziel, V., Pincon-Raymond, M., Olivier, J. L., Chambaz, J. and Pillot, T. (2003) Apoptotic neuronal cell death induced by the non-fibrillar amyloid-beta peptide proceeds through an early reactive oxygen species-dependent cytoskeleton perturbation. J Biol Chem 278: 3437-3445.
43. Sussman, J. L., Harel, M., Frolow, F., Oefner, C., Goldman, A., Toker, L. and Silman, I. (1991) Atomic structure of acetylcholinesterase from Torpedo californica: a prototypic acetylcholine-binding protein. Science 253: 872-879.
44. Tracey, K. J. (2002) The inflammatory reflex. Nature 420: 853-859.
45. Velan, B., Kronman, C., Grosfeld, H., Leitner, M., Gozes, Y., Flashner, Y., Sery, T., Cohen, S., Ben-Aziz, R., Seidman, S, and et al. (1991) Recombinant human acetylcholinesterase is secreted from transiently transfected 293 cells as a soluble globular enzyme. Cell Mol Neurobiol 11: 143-156.
46. Wang, H., Yu, M., Ochani, M., Amella, C. A., Tanovic, M., Susarla, S., Li, J. H., Yang, H., Ulloa, L., Al-Abed, Y., Czura, C. J., and Tracey, K. J. (2003) Nicotinic acetylcholine receptor alpha7 subunit is an essential regulator of inflammation. Nature 421: 384-388.
47. Zhang, W. G., Liu, S. H., Cao, X. M., Cheng, Y. X., Ma, X. R., Yang, Y. and Wang, Y. L. (2005) A phase-I clinical trial of active immunotherapy for acute leukemia using inactivated autologous leukemia cells mixed with IL-2, GM-CSF and IL-6. Leuk Res 29: 3-9.

### CD-ROM CONTENT

The following lists the file content of a duplicate CD-ROM, which is enclosed herewith and filed with the application. These files are incorporated herein by reference and thus form a part of the filed application. File information is provided as: File name/bite size/date of creation/machine format/operating system.

CD-ROM1 (1 file):

1. 28870 Seq List/6289 Kbytes/Jan. 9, 2005/NotePad/PC.

What is claimed is:

1. A BChE derived peptide capable of preventing and/or reversing amyloid fibril formation.

2. The BChE derived peptide of claim 1, selected from the group consisting of SEQ ID NOs:1 and 8-20302.

3. A pharmaceutical composition comprising as an active ingredient BChE or a BChE derived peptide, said peptide being capable of preventing and/or reversing amyloid fibril formation and a pharmaceutically acceptable carrier.

4. The pharmaceutical composition of claim 3, wherein said BChE derived peptide is selected from the group consisting of SEQ ID NOs:1 and 8-20302.

5. The pharmaceutical composition of claim 3, wherein said BChE derived peptide is set forth by SEQ ID NO:1.

6. The pharmaceutical composition of claim 3, wherein said active ingredient is formulated in a therapeutically effective amount providable at a dose range of 0.1-1000 micromol per kg body weight.

7. The pharmaceutical composition of claim 3, wherein said active ingredient is formulated in a therapeutically effective amount providable at a dose range of 1-100 micromol per kg body weight.

8. The pharmaceutical composition of claim 3, wherein said active ingredient is formulated in a therapeutically effective amount providable at a dose range of 5-50 micromol per kg body weight.

9. The pharmaceutical composition of claim 3, wherein said pharmaceutically acceptable carrier is selected for reducing an immugenicity of said peptide.

10. The pharmaceutical composition of claim 3, wherein said pharmaceutically acceptable carrier is selected for sustained release of said peptide.

11. A method of treating an individual having or being predisposed to a disease or disorder associated with amyloid fibril formation, the method comprising administering to the individual a therapeutically effective amount of BChE or BChE derived peptide, thereby treating the individual having or being predisposed to a disease or disorder associated with amyloid fibril formation.

12. The method of claim 11, wherein said BChE derived peptide is selected from the group consisting of SEQ ID NO:1 and 8-20302.

13. The method of claim 11, wherein said disease or disorder associated with amyloid fibril formation is selected from the group consisting of a neurodegenerative disease, a disorder associated with systemic amyloidosis, a disorder associated with localized amyloidosis, a prion disease and/or a polyglutamine disorder.

14. The method of claim 13, wherein said neurodegenerative disease is selected from the group consisting of Alzheimer's disease, Huntington's disease and Parkinson's disease.

15. The method of claim 13, wherein said disorder associated with systemic amyloidosis is selected from the group consisting of Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy.

16. The method of claim 13, wherein said disorder associated with localized amyloidosis is selected from the group consisting of Senile cardiac amyloidosis, Senile cerebral amyloidosis, Endocrine tumors, Medullary carcinoma of thyroid, Type II diabetes and Pancreatic islets β-cells.

17. The method of claim 13, wherein said prion disease is selected from the group consisting of Creutzfeldt-Jakob disease (CJD), spongioform enchephalopathies (TSE's), mad cow disease, Gerstmann-Straussler-Scheinker disease (GSS) and Kuru.

18. The method of claim 13, wherein said polyglutamine disorder is selected from the group consisting of Huntington's disease (HD), Spinal and Bulbar Muscular Atrophy (SBMA), DentatoRubral and PallidoLuysian Atrophy (DRPLA), spinocerebellar ataxia type 1 (SCA1), spinocerebellar ataxia type 2 (SCA2), Spinocerebellar ataxia type-3 (SCA3; Machado-Joseph Disease), Spinocerebellar ataxia type 7 (SCA7) and Spinocerebellar ataxia type 17 (SCA17).

19. The method of claim 11, wherein said amyloid is a protein selected from the group consisting of Transthyretin, Amyloid beta protein, Procalcitonin, LAPP (Amylin), amyloid light chain (AL), non-immunoglobulin amyloid associated (AA), non-immunoglobulin amyloid associated serum precursor (SAA), α-synucleic protein, ataxin and huntingtin.

20. The method of claim 11, wherein said peptide is set forth by SEQ ID NO:1.

21. The method of claim 11, wherein said therapeutically effective amount is providable at a dose range of 0.1-1000 micromol per kg body weight.

22. The method of claim 11, wherein said therapeutically effective amount is providable at a dose range of 1-100 micromol per kg body weight.

23. The method of claim 11, wherein said therapeutically effective amount is providable at a dose range of 5-50 micromol per kg body weight.

24. A method of identifying a BChE derived peptide capable of preventing and/or reversing amyloid fibril formation comprising contacting the BChE derived peptide with an amyloid precursor protein and a β-sheet—responsive dye and measuring a fluorescence intensity resulting from said β-sheet—responsive dye prior to and following said contacting said BChE derived peptide with said amyloid precursor protein, wherein delayed or reduced increase in said fluorescence intensity following said contacting said BChE derived peptide with said amyloid precursor protein is indicative of an ability of the peptide to prevent amyloid fibril formation.

25. The method of claim 24, wherein said BChE derived peptide is selected from the group consisting of SEQ ID NOs:8-20302.

26. The method of claim 24, wherein said β-sheet—responsive dye is a benzothiazole dye.

27. The method of claim 24, wherein said β-sheet—responsive dye is Thioflavin T.

28. The method of claim 27, wherein said Thioflavin T is provided at a concentration range of 0.5-1.5 μM.

29. The method of claim 27, wherein said Thioflavin T is provided at a concentration of about 1 μM.

30. The method of claim 24, wherein said amyloid precursor protein is selected from the group consisting of Transthyretin, Amyloid beta protein, Amyloid beta (1-40), Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

31. The method of claim 30, wherein said Amyloid beta (1-40) is provided at a concentration in the range of 20-50 μM.

32. The method of claim 30, wherein said Amyloid beta (1-40) is provided at a concentration of about 33 μM.

33. A method of preventing and/or reversing amyloid fibril formation in a tissue of an individual comprising increasing a level of BChE or a BChE derived peptide being capable of preventing and/or reversing amyloid fibril formation in the tissue, thereby preventing and/or reversing amyloid fibril formation therein.

34. The method of claim 33, wherein the tissue is of an individual having, or being predisposed to an amyloid fibril-associated disease or disorder.

35. The method of claim 34, wherein said individual has a neurodegenerative disease, a disorder associated with systemic amyloidosis, a disorder associated with localized amyloidosis, a prion disease and/or a polyglutamine disorder.

36. The method of claim 35, wherein said neurodegenerative disease is selected from the group consisting of Alzheimer's disease, Huntington's disease and Parkinson's disease.

37. The method of claim 35, wherein said disorder associated with systemic amyloidosis is selected from the group consisting of Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy.

38. The method of claim 35, wherein said disorder associated with localized amyloidosis is selected from the group consisting of Senile cardiac amyloidosis, Senile cerebral amyloidosis, Endocrine tumors, Medullary carcinoma of thyroid, Type II diabetes and Pancreatic islets β-cells.

39. The method of claim 35, wherein said prion disease is selected from the group consisting of Creutzfeldt-Jakob disease (CJD), spongioform enchephalopathies (TSE's), mad cow disease, Gerstmann-Straussler-Scheinker disease (GSS) and Kuru.

40. The method of claim 35, wherein said polyglutamine disorder is selected from the group consisting of Huntington's disease (HD), Spinal and Bulbar Muscular Atrophy (SBMA), DentatoRubral and PallidoLuysian Atrophy (DRPLA), spinocerebellar ataxia type 1 (SCA1), spinocerebellar ataxia type 2 (SCA2), Spinocerebellar ataxia type-3 (SCA3; Machado-Joseph Disease), Spinocerebellar ataxia type 7 (SCA7) and Spinocerebellar ataxia type 17 (SCA17).

41. The method of claim 33, wherein said amyloid is a protein selected from the group consisting of Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

42. The method of claim 33, wherein said increasing is effected by at least one approach selected from the group consisting of:
(a) expressing in cells of the individual an exogenous polynucleotide encoding said BChE or said BChE derived peptide;
(b) increasing expression of endogenous BChE in the individual;
(c) increasing endogenous BChE activity in the individual;
(d) administering BChE or the BChE derived peptide to the individual; and
(e) administering to the individual cells expressing the BChE or the BChE derived peptide.

43. The method of claim 42, wherein said exogenous polynucleotide encoding said BChE or said BChE derived peptide is derived from SEQ ID NO:7.

44. The method of claim 42, wherein said BChE is as set forth in SEQ ID NO:2.

45. The method of claim 42, wherein said BChE derived peptide is as set forth in any of SEQ ID NOs:1 and 8-20302.

46. A method of treating an individual having or being predisposed to a disease or disorder associated with amyloid fibril formation, the method comprising increasing a level of BChE or a BChE derived peptide in a tissue susceptible to the amyloid fibril formation of the individual, thereby treating the individual having or being predisposed to a disorder associated with amyloid fibril formation.

47. The method of claim 46, wherein said disease or disorder associated with amyloid fibril formation is a neurodegenerative disease, a disorder associated with systemic amyloidosis, a disorder associated with localized amyloidosis, a prion disease and/or a polyglutamine disorder.

48. The method of claim 47, wherein said neurodegenerative disease is selected from the group consisting of Alzheimer's disease, Huntington's disease and Parkinson's disease.

49. The method of claim 47, wherein said disorder associated with systemic amyloidosis is selected from the group consisting of Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy.

50. The method of claim 47, wherein said disorder associated with localized amyloidosis is selected from the group consisting of Senile cardiac amyloidosis, Senile cerebral amyloidosis, Endocrine tumors, Medullary carcinoma of thyroid, Type II diabetes and Pancreatic islets β-cells.

51. The method of claim 47, wherein said prion disease is selected from the group consisting of Creutzfeldt-Jakob disease (CJD), spongioform enchephalopathies (TSE's), mad cow disease, Gerstmann-Straussler-Scheinker disease (GSS) and Kuru.

52. The method of claim 47, wherein said polyglutamine disorder is selected from the group consisting of Huntington's disease (HD), Spinal and Bulbar Muscular Atrophy (SBMA), DentatoRubral and PallidoLuysian Atrophy (DRPLA), spinocerebellar ataxia type 1 (SCA1), spinocerebellar ataxia type 2 (SCA2), Spinocerebellar ataxia type-3 (SCA3; Machado-Joseph Disease), Spinocerebellar ataxia type 7 (SCA7) and Spinocerebellar ataxia type 17 (SCA17).

53. The method of claim 46, wherein said amyloid is a protein selected from the group consisting of Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

54. The method of claim 46, wherein said increasing is effected by at least one approach selected from the group consisting of:
(a) expressing in cells of the individual an exogenous polynucleotide encoding said BChE or said BChE derived peptide;
(b) increasing expression of endogenous BChE in the individual;
(c) increasing endogenous BChE activity in the individual;
(d) administering BChE or the BChE derived peptide to the individual; and
(e) administering to the individual cells expressing the BChE or the BChE derived peptide.

55. The method of claim 54, wherein said exogenous polynucleotide encoding said BChE or said BChE derived peptide is derived from SEQ ID NO:7.

56. The method of claim 54, wherein said BChE is as set forth in SEQ ID NO:2.

57. The method of claim 54, wherein said BChE derived peptide is as set forth in any of SEQ ID NOs:1 and 8-20302.

58. A method of limiting or reducing an inflammatory reaction in an individual, comprising increasing an expression level and/or activity of BChE in the individual, thereby limiting or reducing the inflammatory reaction in the individual.

59. The method of claim 58, wherein the inflammatory reaction is modulated by circulating acetylcholine.

60. The method of claim 58, wherein said individual is subjected to a surgery, stress or a trauma.

61. The method of claim 58, wherein the inflammatory reaction is mediated by at least one pro-inflammatory cytokine selected from the group consisting of IL-1, IL-1α, IL-1β, IL-1ss, IL-6, IL-8, IL-10, IL-12, IL-18 and TNFα.

62. The method of claim 58, wherein said increasing is effected by at least one approach selected from the group consisting of:
(a) expressing in cells of the individual an exogenous polynucleotide encoding at least a functional portion of BChE;
(b) increasing expression of endogenous BChE in the individual;
(c) increasing endogenous BChE activity in the individual;
(d) administering an exogenous polypeptide including at least a functional portion of BChE to the individual;
(e) administering cells expressing the BChE into said individual.

63. The method of claim 62, wherein said exogenous polynucleotide encoding at least a functional portion of BChE is set forth by SEQ ID NO:7.

64. The method of claim 62, wherein said BChE is set forth by SEQ ID NO:2.

65. The use of BChE or a BChE derived peptide in the manufacturing of a medicament for the treatment of a disease or disorder associated with amyloid fibril formation or a predisposition thereto.

66. The use of claim 65, wherein said BChE derived peptide is selected from the group consisting of SEQ ID NO:1 and 8-20302.

67. The use of claim 65, wherein said disease or disorder associated with amyloid fibril formation is selected from the group consisting of a neurodegenerative disease, a disorder associated with systemic amyloidosis, a disorder associated with localized amyloidosis, a prion disease and/or a polyglutamine disorder.

68. The use of claim 67, wherein said neurodegenerative disease is selected from the group consisting of Alzheimer's disease, Huntington's disease and Parkinson's disease.

69. The use of claim 67, wherein said disorder associated with systemic amyloidosis is selected from the group consisting of Multiple myeloma, Chronic inflammatory disease, Rheumatoid arthritis, Tuberculosis, Skin abscess, lung abscess, Cancer, Hodgkin's disease, Hemodialysis for chronic renal failure, Heredofamilial amyloidosis, Familial Mediterranean Fever and Familial amyloid polyneuropathy.

70. The use of claim 67, wherein said disorder associated with localized amyloidosis is selected from the group consisting of Senile cardiac amyloidosis, Senile cerebral amyloidosis, Endocrine tumors, Medullary carcinoma of thyroid, Type II diabetes and Pancreatic islets β-cells.

71. The use of claim 67, wherein said prion disease is selected from the group consisting of Creutzfeldt-Jakob disease (CJD), spongioform enchephalopathies (TSE's), mad cow disease, Gerstmann-Straussler-Scheinker disease (GSS) and Kuru.

72. The use of claim 67, wherein said polyglutamine disorder is selected from the group consisting of Huntington's disease (HD), Spinal and Bulbar Muscular Atrophy (SBMA), DentatoRubral and PallidoLuysian Atrophy (DRPLA), spinocerebellar ataxia type 1 (SCA1), spinocerebellar ataxia type 2 (SCA2), Spinocerebellar ataxia type-3 (SCA3; Machado-Joseph Disease), Spinocerebellar ataxia type 7 (SCA7) and Spinocerebellar ataxia type 17 (SCA17).

73. The use of claim 65, wherein said amyloid is a protein selected from the group consisting of Transthyretin, Amyloid beta protein, Procalcitonin, IAPP (Amylin), amyloid light chain (AL), non-immunoglobulin amyloid associated (AA), non-immunoglobulin amyloid associated serum precursor (SAA), α-synucleic protein, ataxin and huntingtin.

74. The use of claim 65, wherein said BChE derived peptide is set forth by SEQ ID NO:1.

75. The use of claim 65, wherein said medicament is providable at a dose range of 0.1-1000 micromol per kg body weight.

76. The use of claim 65, wherein said medicament is providable at a dose range of 1-100 micromol per kg body weight.

77. The use of claim 65, wherein said medicament is providable at a dose range of 5-50 micromol per kg body weight.
